SEQUENCE LISTING (151 sequences)

SEQ ID NO: 1 - protein, 113 aa, synthetic construct
  REGION 1..113: note = Synthesized
DIVMSQSPSSLAVSVGEKVTMSCKSIQTLLYSSNQKNYLAWFQQKPGQSPKLLIYWASIRKSGVPDRFTGSGSGTDFTLTISSVKAEDLAVYYCQQYYSYPLTFGAGTKLELK

SEQ ID NO: 2 - protein, 107 aa, synthetic construct
  REGION 1..107: note = Synthesized
DIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKLEIK

SEQ ID NO: 3 - protein, 113 aa, synthetic construct
  REGION 1..113: note = Synthesized
DIQMTQSPSSLSASVGDRVTITCKSIQTLLYSSNQKNYLAWFQQKPGKAPKLLIYWASIRKSGVPSRFSGSGSGTDFTLTISSLQPEDFATYYCQQYYSYPLTFGGGTKLEIK

SEQ ID NO: 4 - protein, 113 aa, synthetic construct
  REGION 1..113: note = Synthesized
DIQMTQSPSSLSASVGDRVTITCKSIQTLLYSSNQKNYLAWYQQKPGKAPKLLIYWASIRKSGVPSRFSGSGSGTDFTLTISSLQPEDFATYYCQQYYSYPLTFGGGTKLEIK

SEQ ID NO: 5 - protein, 113 aa, synthetic construct
  REGION 1..113: note = Synthesized
DIQMTQSPSSLSASVGDRVTITCKSIQTLLYSSNQKNYLAWFQQKPGKAPKLLIYWASIRKSGVPSRFSGSGSGTDFTLTISSLQPEDLATYYCQQYYSYPLTFGGGTKLEIK

SEQ ID NO: 6 - protein, 116 aa, synthetic construct
  REGION 1..116: note = Synthesized
EVKLVESGGGLVKPGASLKLSCAASGFTFSNYGMSWVRQTSDKRLEWVASISSGGGSTYYPDNVKGRFTISREDAKNTLYLQMSSLRSEDTALYYCSRGGAGIDYWGQGTTLTVSS

SEQ ID NO: 7 - protein, 116 aa, synthetic construct
  REGION 1..116: note = Synthesized
EVQLVESGGGLVQPGGSLRLSCAASGFTFSSYWMSWVRQAPGKGLEWVANIKQDGSEKYYVDSVKGRFTISRDNAKNSLYLQMNSLRAEDTAVYYCARGSSDMDYWGQGTLVTVSS

SEQ ID NO: 8 - protein, 116 aa, synthetic construct
  REGION 1..116: note = Synthesized
EVQLVESGGGLVQPGGSLRLSCAASGFTFSNYGMSWVRQAPGKGLEWVASISSGGGSTYYPDNVKGRFTISRDDAKNSLYLQMNSLRAEDTAVYYCSRGGAGIDYWGQGTLVTVSS

SEQ ID NO: 9 - protein, 116 aa, synthetic construct
  REGION 1..116: note = Synthesized
EVQLVESGGGLVQPGGSLRLSCAASGFTFSNYGMSWVRQAPGKGLEWVASISSGGGSTYYPDNVKGRFTISRDNAKNSLYLQMNSLRAEDTAVYYCSRGGAGIDYWGQGTLVTVSS

SEQ ID NO: 10 - protein, 116 aa, synthetic construct
  REGION 1..116: note = Synthesized
EVQLVESGGGLVQPGGSLRLSCAASGFTFSNYGMSWVRQAPGKGLEWVASISSGGGSTYYPDNVKGRFTISRDDAKNSLYLQMNSLRAEDTAVYYCARGGAGIDYWGQGTLVTVSS

SEQ ID NO: 11 - protein, 116 aa, synthetic construct
  REGION 1..116: note = Synthesized
EVQLVESGGGLVQPGGSLRLSCAASGFTFSNYGMSWVRQAPGKGLEWVASISSGGGSTYYPDNVKGRFTISRDNAKNSLYLQMNSLRAEDTAVYYCARGGAGIDYWGQGTLVTVSS

SEQ ID NO: 12 - protein, 140 aa, Homo sapiens
MDVFMKGLSKAKEGVVAAAEKTKQGVAEAAGKTKEGVLYVGSKTKEGVVHGVATVAEKTKEQVTNVGGAVVTGVTAVAQKTVEGAGSIAAATGFVKKDQLGKNEEGAPQEGILEDMPVDPDNEAYEMPSEEGYQDYEPEA

SEQ ID NO: 13 - protein, 107 aa, synthetic construct [sequence omitted]

SEQ ID NO: 14 - DNA, 324 bp, synthetic construct
  misc_feature 1..324: note = Synthesized
  source 1..324: mol_type = other DNA
cgaaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagctcaacaggggagagtgttag

SEQ ID NO: 15 - protein, 330 aa, synthetic construct
  REGION 1..330: note = Synthesized
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNVKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - DNA, 991 bp, synthetic construct
  misc_feature 1..991: note = Synthesized
  source 1..991: mol_type = other DNA
gcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagaccacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgtcaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacacgctgcccccatcccgggaggagatgaccaagaaccaggtcagcctgacctgcctggtcaaaggcttctatcccagcgacatcgcgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaatga

SEQ ID NO: 17 - DNA, 339 bp, synthetic construct
  misc_feature 1..339: note = Synthesized
  source 1..339: mol_type = other DNA
gacatccagatgacccagtccccctcctccctgtccgcctccgtgggcgaccgcgtgaccatcacctgcaagtccatccagaccctgctgtactcctccaaccagaagaactacctggcctggttccagcagaagcccggcaaggcccccaagctgctgatctactgggcctccatccgcaagtccggcgtgccctcccgcttctccggctccggctccggcaccgacttcaccctgaccatctcctccctgcagcccgaggacttcgccacctactactgccagcagtactactcctaccccctgaccttcggcggcggcaccaagctggagatcaag

SEQ ID NO: 18 - DNA, 339 bp, synthetic construct
  misc_feature 1..339: note = Synthesized
  source 1..339: mol_type = other DNA
gacatccagatgacccagtccccctcctccctgtccgcctccgtgggcgaccgcgtgaccatcacctgcaagtccatccagaccctgctgtactcctccaaccagaagaactacctggcctggtaccagcagaagcccggcaaggcccccaagctgctgatctactgggcctccatccgcaagtccggcgtgccctcccgcttctccggctccggctccggcaccgacttcaccctgaccatctcctccctgcagcccgaggacttcgccacctactactgccagcagtactactcctaccccctgaccttcggcggcggcaccaagctggagatcaag

SEQ ID NO: 19 - DNA, 339 bp, synthetic construct
  misc_feature 1..339: note = Synthesized
  source 1..339: mol_type = other DNA
gacatccagatgacccagtccccctcctccctgtccgcctccgtgggcgaccgcgtgaccatcacctgcaagtccatccagaccctgctgtactcctccaaccagaagaactacctggcctggttccagcagaagcccggcaaggcccccaagctgctgatctactgggcctccatccgcaagtccggcgtgccctcccgcttctccggctccggctccggcaccgacttcaccctgaccatctcctccctgcagcccgaggacctggccacctactactgccagcagtactactcctaccccctgaccttcggcggcggcaccaagctggagatcaag

SEQ ID NO: 20 - DNA, 348 bp, synthetic construct
  misc_feature 1..348: note = Synthesized
  source 1..348: mol_type = other DNA
gaggtgcagctggtggagtccggcggcggcctggtgcagcccggcggctccctgcgcctgtcctgcgccgcctccggcttcaccttctccaactacggcatgtcctgggtgcgccaggcccccggcaagggcctggagtgggtggcctccatctcctccggcggcggctccacctactaccccgacaacgtgaagggccgcttcaccatctcccgcgacgacgccaagaactccctgtacctgcagatgaactccctgcgcgccgaggacaccgccgtgtactactgctcccgcggcggcgccggcatcgactactggggccagggcaccctggtgaccgtgtcctcc

SEQ ID NO: 21 - DNA, 348 bp, synthetic construct
  misc_feature 1..348: note = Synthesized
  source 1..348: mol_type = other DNA
gaggtgcagctggtggagtccggcggcggcctggtgcagcccggcggctccctgcgcctgtcctgcgccgcctccggcttcaccttctccaactacggcatgtcctgggtgcgccaggcccccggcaagggcctggagtgggtggcctccatctcctccggcggcggctccacctactaccccgacaacgtgaagggccgcttcaccatctcccgcgacaacgccaagaactccctgtacctgcagatgaactccctgcgcgccgaggacaccgccgtgtactactgctcccgcggcggcgccggcatcgactactggggccagggcaccctggtgaccgtgtcctcc

SEQ ID NO: 22 - DNA, 348 bp, synthetic construct
  misc_feature 1..348: note = Synthesized
  source 1..348: mol_type = other DNA
gaggtgcagctggtggagtccggcggcggcctggtgcagcccggcggctccctgcgcctgtcctgcgccgcctccggcttcaccttctccaactacggcatgtcctgggtgcgccaggcccccggcaagggcctggagtgggtggcctccatctcctccggcggcggctccacctactaccccgacaacgtgaagggccgcttcaccatctcccgcgacgacgccaagaactccctgtacctgcagatgaactccctgcgcgccgaggacaccgccgtgtactactgcgcccgcggcggcgccggcatcgactactggggccagggcaccctggtgaccgtgtcctcc

SEQ ID NO: 23 - DNA, 348 bp, synthetic construct
  misc_feature 1..348: note = Synthesized
  source 1..348: mol_type = other DNA
gaggtgcagctggtggagtccggcggcggcctggtgcagcccggcggctccctgcgcctgtcctgcgccgcctccggcttcaccttctccaactacggcatgtcctgggtgcgccaggcccccggcaagggcctggagtgggtggcctccatctcctccggcggcggctccacctactaccccgacaacgtgaagggccgcttcaccatctcccgcgacaacgccaagaactccctgtacctgcagatgaactccctgcgcgccgaggacaccgccgtgtactactgcgcccgcggcggcgccggcatcgactactggggccagggcaccctggtgaccgtgtcctcc

SEQ ID NO: 24 - protein, 22 aa, synthetic construct
  REGION 1..22: note = Synthesized
MDMRVPAQLLGLLMLWVSGSSG

SEQ ID NO: 25 - DNA, 66 bp, synthetic construct
  misc_feature 1..66: note = Synthesized
  source 1..66: mol_type = other DNA
atggacatgcgcgtgcccgcccagctgctgggcctgctgatgctgtgggtgtccggctcctccggc

SEQ ID NO: 26 - protein, 19 aa, synthetic construct
  REGION 1..19: note = Synthesized
MEFGLSWLFLVAILKGVQC

SEQ ID NO: 27 - DNA, 57 bp, synthetic construct
  misc_feature 1..57: note = Synthesized
  source 1..57: mol_type = other DNA
atggagttcggcctgtcctggctgttcctggtggccatcctgaagggcgtgcagtgc

SEQ ID NO: 28 - protein, 113 aa, synthetic construct
  REGION 1..113: note = Synthesized
  VARIANT 42: note = Xaa = Y or F
  VARIANT 89: note = Xaa = F or L
DIQMTQSPSSLSASVGDRVTITCKSIQTLLYSSNQKNYLAWXQQKPGKAPKLLIYWASIRKSGVPSRFSGSGSGTDFTLTISSLQPEDXATYYCQQYYSYPLTFGGGTKLEIK

SEQ ID NO: 29 - protein, 116 aa, synthetic construct
  REGION 1..116: note = Synthesized
  VARIANT 74: note = Xaa = N or D
  VARIANT 97: note = Xaa = A or S
EVQLVESGGGLVQPGGSLRLSCAASGFTFSNYGMSWVRQAPGKGLEWVASISSGGGSTYYPDNVKGRFTISRDXAKNSLYLQMNSLRAEDTAVYYCXRGGAGIDYWGQGTLVTVSS

SEQ ID NO: 30 - protein, 106 aa, synthetic construct
  REGION 1..106: note = Synthesized
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 31 - DNA, 321 bp, synthetic construct
  misc_feature 1..321: note = Synthesized
  source 1..321: mol_type = other DNA
aactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagctcaacaggggagagtgttag

SEQ ID NO: 32 - protein, 220 aa, synthetic construct
  REGION 1..220: note = Synthesized
DIQMTQSPSSLSASVGDRVTITCKSIQTLLYSSNQKNYLAWFQQKPGKAPKLLIYWASIRKSGVPSRFSGSGSGTDFTLTISSLQPEDLATYYCQQYYSYPLTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 33 - protein, 219 aa, synthetic construct
  REGION 1..219: note = Synthesized
DIQMTQSPSSLSASVGDRVTITCKSIQTLLYSSNQKNYLAWFQQKPGKAPKLLIYWASIRKSGVPSRFSGSGSGTDFTLTISSLQPEDLATYYCQQYYSYPLTFGGGTKLEIKTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 34 - protein, 446 aa, synthetic construct
  REGION 1..446: note = Synthesized
EVQLVESGGGLVQPGGSLRLSCAASGFTFSNYGMSWVRQAPGKGLEWVASISSGGGSTYYPDNVKGRFTISRDDAKNSLYLQMNSLRAEDTAVYYCARGGAGIDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNVKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 35 - protein, 330 aa, synthetic construct
  REGION 1..330: note = Synthesized
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 36 - protein, 446 aa, synthetic construct; identical to SEQ ID NO: 34
  REGION 1..446: note = Synthesized

SEQ ID NO: 37 - protein, 446 aa, synthetic construct
  REGION 1..446: note = Synthesized
EVQLVESGGGLVQPGGSLRLSCAASGFTFSNYGMSWVRQAPGKGLEWVASISSGGGSTYYPDNVKGRFTISRDDAKNSLYLQMNSLRAEDTAVYYCARGGAGIDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 38 - DNA, 345 bp, synthetic construct
  misc_feature 1..345: note = Synthesized
  source 1..345: mol_type = other DNA
caggtccagctgcagcagtctggggctgaactggcaaaacctgggacctcagtgcagatgtcctgcaaggcttctggctacacctttactaattactggatgaactggataaaagcgaggcctggacagggtctggaatggattggggctactaatcctaacaatggttatactgactacaatcagaggttcaaggacaaggccatattaactgcagacaaatcctccaatacagcctacatgcacctgagcagcctgacatctgaagactctgcagtctatttctgtgcaagtggggggcacttggcttactggggccaggggactgtggtcactgtctctgca

SEQ ID NO: 39 - protein, 115 aa, synthetic construct
  REGION 1..115: note = Synthesized
QVQLQQSGAELAKPGTSVQMSCKASGYTFTNYWMNWIKARPGQGLEWIGATNPNNGYTDYNQRFKDKAILTADKSSNTAYMHLSSLTSEDSAVYFCASGGHLAYWGQGTVVTVSA

SEQ ID NO: 40 - DNA, 57 bp, synthetic construct
  misc_feature 1..57: note = Synthesized
  source 1..57: mol_type = other DNA
atggaaaggcactggatctttctcttcctgttatcagtaactggaggtgtccactcc

SEQ ID NO: 41 - protein, 19 aa, synthetic construct
  REGION 1..19: note = Synthesized
MERHWIFLFLLSVTGGVHS

SEQ ID NO: 42 - DNA, 336 bp, synthetic construct
  misc_feature 1..336: note = Synthesized
  source 1..336: mol_type = other DNA
gatgttgtgatgacccaaattccactctacctgtctgtcagtcctggagatcaagcctccatctcttgcagatctagtcagagccttttccatagtaaaggaaacacctatttacattggtatctgcagaagccaggccagtctccaaagctcctgatcaacagggtttccaaccgattttctggggtcccagacaggttcagtggcagtggatcagggacagatttcacactcaagatcagcggagtggaggctgaagatctgggagtttatttctgttctcaaagtgcacatgttccgtggacgttcggtggaggcaccaagctggaaatcaga

SEQ ID NO: 43 - protein, 112 aa, synthetic construct
  REGION 1..112: note = Synthesized
DVVMTQIPLYLSVSPGDQASISCRSSQSLFHSKGNTYLHWYLQKPGQSPKLLINRVSNRFSGVPDRFSGSGSGTDFTLKISGVEAEDLGVYFCSQSAHVPWTFGGGTKLEIR

SEQ ID NO: 44 - DNA, 60 bp, synthetic construct
  misc_feature 1..60: note = Synthesized
  source 1..60: mol_type = other DNA
atgaagttgcctgttaggctgttggtgctgatgttctggattcctgcttccagcagtgat

SEQ ID NO: 45 - protein, 20 aa, synthetic construct
  REGION 1..20: note = Synthesized
MKLPVRLLVLMFWIPASSSD

SEQ ID NO: 46 - protein, 12 aa, synthetic construct
  REGION 1..12: note = Synthesized
VDPDNEAYEGGC

SEQ ID NO: 47 - protein, 5 aa, synthetic construct
  REGION 1..5: note = Synthesized
NYWMN

SEQ ID NO: 48 - protein, 17 aa, synthetic construct
  REGION 1..17: note = Synthesized
ATNPNNGYTDYNQRFKD

SEQ ID NO: 49 - protein, 6 aa, synthetic construct
  REGION 1..6: note = Synthesized
GGHLAY

SEQ ID NO: 50 - protein, 16 aa, synthetic construct
  REGION 1..16: note = Synthesized
RSSQSLFHSKGNTYLH

SEQ ID NO: 51 - protein, 7 aa, synthetic construct
  REGION 1..7: note = Synthesized
RVSNRFS

SEQ ID NO: 52 - protein, 9 aa, synthetic construct
  REGION 1..9: note = Synthesized
SQSAHVPWT

SEQ ID NO: 53 - DNA, 402 bp, synthetic construct
  misc_feature 1..402: note = Synthesized
  source 1..402: mol_type = other DNA
atggaaaggcactggatctttctcttcctgttatcagtaactggaggtgtccactcccaggtccagctgcagcagtctggggctgaactggcaaaacctgggacctcagtgcagatgtcctgcaaggcttctggctacacctttactaattactggatgaactggataaaagcgaggcctggacagggtctggaatggattggggctactaatcctaacaatggttatactgactacaatcagaggttcaaggacaaggccatattaactgcagacaaatcctccaatacagcctacatgcacctgagcagcctgacatctgaagactctgcagtctatttctgtgcaagtggggggcacttggcttactggggccaggggactgtggtcactgtctctgca

SEQ ID NO: 54 - protein, 134 aa, synthetic construct
  REGION 1..134: note = Synthesized
MERHWIFLFLLSVTGGVHSQVQLQQSGAELAKPGTSVQMSCKASGYTFTNYWMNWIKARPGQGLEWIGATNPNNGYTDYNQRFKDKAILTADKSSNTAYMHLSSLTSEDSAVYFCASGGHLAYWGQGTVVTVSA

SEQ ID NO: 55 - DNA, 393 bp, synthetic construct
  misc_feature 1..393: note = Synthesized
  source 1..393: mol_type = other DNA
atgaagttgcctgttaggctgttggtgctgatgttctggattcctgcttccagcagtgatgttgtgatgacccaaattccactctacctgtctgtcagtcctggagatcaagcctccatctcttgcagatctagtcagagccttttccatagtaaaggaaacacctatttacattggtatctgcagaagccaggccagtctccaaagctcctgatcaacagggtttccaaccgattttctggggtcccagacaggttcagtggcagtggatcagggacagatttcacactcaagatcagcggagtggaggctgaagatctgggagtttatttctgttctcaaagtgcacatgttccgtggacgttcggtggaggcaccaagctggaaatcaga

SEQ ID NO: 56 - protein, 131 aa, synthetic construct
  REGION 1..131: note = Synthesized
MKLPVRLLVLMFWIPASSSDVVMTQIPLYLSVSPGDQASISCRSSQSLFHSKGNTYLHWYLQKPGQSPKLLINRVSNRFSGVPDRFSGSGSGTDFTLKISGVEAEDLGVYFCSQSAHVPWTFGGGTKLEIR

SEQ ID NO: 57 - protein, 118 aa, synthetic construct
  REGION 1..118: note = Synthesized
QVQLVQSGAEVKKPGSSVKVSCKASGGTFNNYAINWVRQAPGQGLEWMGGIIPIFGTTTYAQKFQGRVTITADESTNTAYMELSSLRSEDTAVYYCAREGNLNWLDPWGQGTLVTVSS

SEQ ID NO: 58 - protein, 115 aa, synthetic construct
  REGION 1..115: note = Synthesized
QVQLVQSGAELKKPGSSVKVSCKASGYTFTNYWMNWVRQAPGQGLEWIGATNPNNGYTDYNQRFKDRATLTADKSTNTAYMELSSLRSEDTAVYYCARGGHLAYWGQGTLVTVSS

SEQ ID NO: 59 - protein, 115 aa, synthetic construct
  REGION 1..115: note = Synthesized
QVQLVQSGAELKKPGSSVKVSCKASGYTFTNYWMNWVRQAPGQGLEWIGATNPNNGYTDYNQRFKDRVTITADKSTNTAYMELSSLRSEDTAVYYCARGGHLAYWGQGTLVTVSS

SEQ ID NO: 60 - protein, 115 aa, synthetic construct
  REGION 1..115: note = Synthesized
QVQLVQSGAELKKPGSSVKVSCKASGYTFTNYWMNWVRQAPGQGLEWIGATNPNNGYTDYNQRFKDRATLTADKSTNTAYMELSSLRSEDTAVYFCASGGHLAYWGQGTLVTVSS

SEQ ID NO: 61 - protein, 115 aa, synthetic construct
  REGION 1..115: note = Synthesized
QVQLVQSGAELKKPGSSVKVSCKASGYTFTNYWMNWVRQAPGQGLEWIGATNPNNGYTDYNQRFKDRATLTADKSTNTAYMELSSLRSEDTAVYYCASGGHLAYWGQGTLVTVSS

SEQ ID NO: 62 - protein, 115 aa, synthetic construct
  REGION 1..115: note = Synthesized
QVQLVQSGAELKKPGSSVKVSCKASGYTFTNYWMNWVRQAPGQGLEWIGATNPNNGYTDYNQRFKDRVTITADKSTNTAYMELSSLRSEDTAVYYCASGGHLAYWGQGTLVTVSS

SEQ ID NO: 63 - DNA, 402 bp, synthetic construct
  misc_feature 1..402: note = Synthesized
  source 1..402: mol_type = other DNA
atggagttcggcctgtcctggctgttcctggtggccatcctgaagggcgtgcagtgccaggtgcagctggtgcagtccggcgccgagctgaagaagcccggctcctccgtgaaggtgtcctgcaaggcctccggctacaccttcaccaactactggatgaactgggtgcgccaggcccccggccagggcctggagtggatcggcgccaccaaccccaacaacggctacaccgactacaaccagcgcttcaaggaccgcgccaccctgaccgccgacaagtccaccaacaccgcctacatggagctgtcctccctgcgctccgaggacaccgccgtgtactactgcgcccgcggcggccacctggcctactggggccagggcaccctggtgaccgtgtcctcc

SEQ ID NO: 64 - DNA, 402 bp, synthetic construct
  misc_feature 1..402: note = Synthesized
  source 1..402: mol_type = other DNA
atggagttcggcctgtcctggctgttcctggtggccatcctgaagggcgtgcagtgccaggtgcagctggtgcagtccggcgccgagctgaagaagcccggctcctccgtgaaggtgtcctgcaaggcctccggctacaccttcaccaactactggatgaactgggtgcgccaggcccccggccagggcctggagtggatcggcgccaccaaccccaacaacggctacaccgactacaaccagcgcttcaaggaccgcgtgaccatcaccgccgacaagtccaccaacaccgcctacatggagctgtcctccctgcgctccgaggacaccgccgtgtactactgcgcccgcggcggccacctggcctactggggccagggcaccctggtgaccgtgtcctcc

SEQ ID NO: 65 - DNA, 402 bp, synthetic construct
  misc_feature 1..402: note = Synthesized
  source 1..402: mol_type = other DNA
atggagttcggcctgtcctggctgttcctggtggccatcctgaagggcgtgcagtgccaggtgcagctggtgcagtccggcgccgagctgaagaagcccggctcctccgtgaaggtgtcctgcaaggcctccggctacaccttcaccaactactggatgaactgggtgcgccaggcccccggccagggcctggagtggatcggcgccaccaaccccaacaacggctacaccgactacaaccagcgcttcaaggaccgcgccaccctgaccgccgacaagtccaccaacaccgcctacatggagctgtcctccctgcgctccgaggacaccgccgtgtacttctgcgcctccggcggccacctggcctactggggccagggcaccctggtgaccgtgtcctcc

SEQ ID NO: 66 - DNA, 402 bp, synthetic construct
  misc_feature 1..402: note = Synthesized
  source 1..402: mol_type = other DNA
atggagttcggcctgtcctggctgttcctggtggccatcctgaagggcgtgcagtgccaggtgcagctggtgcagtccggcgccgagctgaagaagcccggctcctccgtgaaggtgtcctgcaaggcctccggctacaccttcaccaactactggatgaactgggtgcgccaggcccccggccagggcctggagtggatcggcgccaccaaccccaacaacggctacaccgactacaaccagcgcttcaaggaccgcgccaccctgaccgccgacaagtccaccaacaccgcctacatggagctgtcctccctgcgctccgaggacaccgccgtgtactactgcgcctccggcggccacctggcctactggggccagggcaccctggtgaccgtgtcctcc

SEQ ID NO: 67 - DNA, 402 bp, synthetic construct
  misc_feature 1..402: note = Synthesized
  source 1..402: mol_type = other DNA
atggagttcggcctgtcctggctgttcctggtggccatcctgaagggcgtgcagtgccaggtgcagctggtgcagtccggcgccgagctgaagaagcccggctcctccgtgaaggtgtcctgcaaggcctccggctacaccttcaccaactactggatgaactgggtgcgccaggcccccggccagggcctggagtggatcggcgccaccaaccccaacaacggctacaccgactacaaccagcgcttcaaggaccgcgtgaccatcaccgccgacaagtccaccaacaccgcctacatggagctgtcctccctgcgctccgaggacaccgccgtgtactactgcgccagcggcggccacctggcctactggggccagggcaccctggtgaccgtgtcctcc

SEQ ID NO: 68 - protein, 112 aa, synthetic construct
  REGION 1..112: note = Synthesized
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTPPTFGGGTKVEIK

SEQ ID NO: 69 - protein, 112 aa, synthetic construct
  REGION 1..112: note = Synthesized
DVVMTQSPLSLSVSPGEPASISCRSSQSLFHSKGNTYLHWYLQKPGQSPKLLINRVSNRFSGVPDRFSGSGSGTDFTLKISRVEAEDVGVYFCSQSAHVPWTFGGGTKVEIK

SEQ ID NO: 70 - protein, 112 aa, synthetic construct
  REGION 1..112: note = Synthesized
DIVMTQSPLSLSVSPGEPASISCRSSQSLFHSKGNTYLHWYLQKPGQSPKLLIYRVSNRFSGVPDRFSGSGSGTDFTLKISRVEAEDVGVYFCSQSAHVPWTFGGGTKVEIK

SEQ ID NO: 71 - protein, 112 aa, synthetic construct
  REGION 1..112: note = Synthesized
DVVMTQSPLSLSVSPGEPASISCRSSQSLFHSKGNTYLHWYLQKPGQSPQLLINRVSNRFSGVPDRFSGSGSGTDFTLKISRVEAEDVGVYFCSQSAHVPWTFGGGTKVEIK

SEQ ID NO: 72 - protein, 112 aa, synthetic construct
  REGION 1..112: note = Synthesized
DIVMTQSPLSLSVSPGEPASISCRSSQSLFHSKGNTYLHWYLQKPGQSPQLLIYRVSNRFSGVPDRFSGSGSGTDFTLKISRVEAEDVGVYFCSQSAHVPWTFGGGTKVEIK

SEQ ID NO: 73 - DNA, 402 bp, synthetic construct
  misc_feature 1..402: note = Synthesized
  source 1..402: mol_type = other DNA
atggacatgcgcgtgcccgcccagctgctgggcctgctgatgctgtgggtgtccggctcctccggcgacgtggtgatgacccagtcccccctgtccctgtccgtgtcccccggcgagcccgcctccatctcctgccgctcctcccagtccctgttccactccaagggcaacacctacctgcactggtacctgcagaagcccggccagtcccccaagctgctgatcaaccgcgtgtccaaccgcttctccggcgtgcccgaccgcttctccggctccggctccggcaccgacttcaccctgaagatctcccgcgtggaggccgaggacgtgggcgtgtacttctgctcccagtccgcccacgtgccctggaccttcggcggcggcaccaaggtggagatcaag

SEQ ID NO: 74 - DNA, 402 bp, synthetic construct
  misc_feature 1..402: note = Synthesized
  source 1..402: mol_type = other DNA
atggacatgcgcgtgcccgcccagctgctgggcctgctgatgctgtgggtgtccggctcctccggcgacatcgtgatgacccagtcccccctgtccctgtccgtgtcccccggcgagcccgcctccatctcctgccgctcctcccagtccctgttccactccaagggcaacacctacctgcactggtacctgcagaagcccggccagtcccccaagctgctgatctaccgcgtgtccaaccgcttctccggcgtgcccgaccgcttctccggctccggctccggcaccgacttcaccctgaagatctcccgcgtggaggccgaggacgtgggcgtgtactactgctcccagtccgcccacgtgccctggaccttcggcggcggcaccaaggtggagatcaag

SEQ ID NO: 75 - DNA, 402 bp, synthetic construct
  misc_feature 1..402: note = Synthesized
  source 1..402: mol_type = other DNA
atggacatgcgcgtgcccgcccagctgctgggcctgctgatgctgtgggtgtccggctcctccggcgacgtggtgatgacccagtcccccctgtccctgtccgtgtcccccggcgagcccgcctccatctcctgccgctcctcccagtccctgttccactccaagggcaacacctacctgcactggtacctgcagaagcccggccagtccccccagctgctgatcaaccgcgtgtccaaccgcttctccggcgtgcccgaccgcttctccggctccggctccggcaccgacttcaccctgaagatctcccgcgtggaggccgaggacgtgggcgtgtacttctgctcccagtccgcccacgtgccctggaccttcggcggcggcaccaaggtggagatcaag

SEQ ID NO: 76 - DNA, 402 bp, synthetic construct
  misc_feature 1..402: note = Synthesized
  source 1..402: mol_type = other DNA
atggacatgcgcgtgcccgcccagctgctgggcctgctgatgctgtgggtgtccggctcctccggcgacatcgtgatgacccagtcccccctgtccctgtccgtgtcccccggcgagcccgcctccatctcctgccgctcctcccagtccctgttccactccaagggcaacacctacctgcactggtacctgcagaagcccggccagtccccccagctgctgatctaccgcgtgtccaaccgcttctccggcgtgcccgaccgcttctccggctccggctccggcaccgacttcaccctgaagatctcccgcgtggaggccgaggacgtgggcgtgtactactgctcccagtccgcccacgtgccctggaccttcggcggcggcaccaaggtggagatcaag

SEQ ID NO: 77 - DNA, 321 bp, synthetic construct
  misc_feature 1..321: note = Synthesized
  source 1..321: mol_type = other DNA
actgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 78 - protein, 35 aa, synthetic construct
  REGION 1..35: note = Synthesized
EQVTNVGGAVVTGVTAVAQKTVEGAGSIAAATGFV

SEQ ID NO: 79 - protein, 28 aa, synthetic construct
  REGION 1..28: note = Synthesized
KEQVTNVGGAVVTGVTAVAQKTVEGAGS

SEQ ID NO: 80 - DNA, 405 bp, synthetic construct
  misc_feature 1..405: note = Synthesized
  source 1..405: mol_type = other DNA
atgggatggagctgggtctttatcttcctcctgtcaggaactgcaggtgtccattgccaggtccagctgcagcagtctggacctgagctggtgaagcctgggacttcagtgaagatatcctgcaaggcttctggctacagcttcacaagctactatatacactgggtgaagcagagtcctggacagggacttgagtggattggatggatttatcctggaagtggtaatactaagtacagtgagaagttcaagggcaaggccacactgactgcagacacatcctccagcacagcctacatgcagctcagcagcctgacatctgaggactctgcagtctatttctgtgcaagagatggttgctacgggtttgcttactggggccaagggactctggtcactgtctct

SEQ ID NO: 81 - protein, 135 aa, synthetic construct
  REGION 1..135: note = Synthesized
MGWSWVFIFLLSGTAGVHCQVQLQQSGPELVKPGTSVKISCKASGYSFTSYYIHWVKQSPGQGLEWIGWIYPGSGNTKYSEKFKGKATLTADTSSSTAYMQLSSLTSEDSAVYFCARDGCYGFAYWGQGTLVTVS

SEQ ID NO: 82 - DNA, 393 bp, synthetic construct
  misc_feature 1..393: note = Synthesized
  source 1..393: mol_type = other DNA
atggagacagacacactcctgttatgggtgctgctgctctgggttccaggctccactggtgacattgtgctgacccaatctccagcttctttggctgtgtctctagggcagagggccaccatctcctgcaaggccagccaaagtgttgattatgatggtgatagttatatgaactggtaccaacagaaaccaggacagccacccaaattcctcatctgtgctgcatccaatctagaatctgggatcccagccaggtttagtggcagtgggtctgggacagacttcaccctcaacatccatcctgtggaggaggaggatgctgcaacctattactgtcagcaaagtaatgaggatccattcacgttcggctcggggacaaagttggaaataaaa

SEQ ID NO: 83 - protein, 131 aa, synthetic construct
  REGION 1..131: note = Synthesized
METDTLLLWVLLLWVPGSTGDIVLTQSPASLAVSLGQRATISCKASQSVDYDGDSYMNWYQQKPGQPPKFLICAASNLESGIPARFSGSGSGTDFTLNIHPVEEEDAATYYCQQSNEDPFTFGSGTKLEIK

SEQ ID NO: 84 - DNA, 345 bp, synthetic construct
  misc_feature 1..345: note = Synthesized
  source 1..345: mol_type = other DNA
gtccagctgcagcagtctggacctgagctggtgaagcctgggacttcagtgaagatatcctgcaaggcttctggctacagcttcacaagctactatatacactgggtgaagcagagtcctggacagggacttgagtggattggatggatttatcctggaagtggtaatactaagtacagtgagaagttcaagggcaaggccacactgactgcagacacatcctccagcacagcctacatgcagctcagcagcctgacatctgaggactctgcagtctatttctgtgcaagagatggttgctacgggtttgcttactggggccaagggactctggtcactgtctct

SEQ ID NO: 85 - protein, 115 aa, synthetic construct
  REGION 1..115: note = Synthesized
VQLQQSGPELVKPGTSVKISCKASGYSFTSYYIHWVKQSPGQGLEWIGWIYPGSGNTKYSEKFKGKATLTADTSSSTAYMQLSSLTSEDSAVYFCARDGCYGFAYWGQGTLVTVS

SEQ ID NO: 86 - DNA, 333 bp, synthetic construct
  misc_feature 1..333: note = Synthesized
  source 1..333: mol_type = other DNA
gacattgtgctgacccaatctccagcttctttggctgtgtctctagggcagagggccaccatctcctgcaaggccagccaaagtgttgattatgatggtgatagttatatgaactggtaccaacagaaaccaggacagccacccaaattcctcatctgtgctgcatccaatctagaatctgggatcccagccaggtttagtggcagtgggtctgggacagacttcaccctcaacatccatcctgtggaggaggaggatgctgcaacctattactgtcagcaaagtaatgaggatccattcacgttcggctcggggacaaagttggaaataaaa

SEQ ID NO: 87 - protein, 111 aa, synthetic construct
  REGION 1..111: note = Synthesized
DIVLTQSPASLAVSLGQRATISCKASQSVDYDGDSYMNWYQQKPGQPPKFLICAASNLESGIPARFSGSGSGTDFTLNIHPVEEEDAATYYCQQSNEDPFTFGSGTKLEIK

SEQ ID NO: 88 - protein, 5 aa, synthetic construct
  REGION 1..5: note = Synthesized
SYYIH

SEQ ID NO: 89 - protein, 17 aa, synthetic construct
  REGION 1..17: note = Synthesized
WIYPGSGNTKYSEKFKG

SEQ ID NO: 90 - protein, 8 aa, synthetic construct
  REGION 1..8: note = Synthesized
DGCYGFAY

SEQ ID NO: 91 - protein, 15 aa, synthetic construct
  REGION 1..15: note = Synthesized
KASQSVDYDGDSYMN

SEQ ID NO: 92 - protein, 7 aa, synthetic construct
  REGION 1..7: note = Synthesized
AASNLES

SEQ ID NO: 93 - protein, 9 aa, synthetic construct
  REGION 1..9: note = Synthesized
QQSNEDPFT

SEQ ID NO: 94 - DNA, 351 bp, synthetic construct
  misc_feature 1..351: note = Synthesized
  source 1..351: mol_type = other DNA
caggtgcagctggtgcagtccggcgccgagctgaagaagcccggcgcctccgtgaaggtgtcctgcaaggcctccggctactccttcacctcctactacatccactgggtgaagcaggcccccggccagggcctggagtggatcggctggatctaccccggctccggcaacaccaagtactccgagaagttcaagggccgcgccaccctgaccgccgacacctccacctccaccgcctacatggagctgcgctccctgcgctccgacgacaccgccgtgtacttctgcgcccgcgacggctgctacggcttcgcctactggggccagggcaccctggtgaccgtgtcctca

SEQ ID NO: 95 - protein, 117 aa, synthetic construct
  REGION 1..117: note = Synthesized
QVQLVQSGAELKKPGASVKVSCKASGYSFTSYYIHWVKQAPGQGLEWIGWIYPGSGNTKYSEKFKGRATLTADTSTSTAYMELRSLRSDDTAVYFCARDGCYGFAYWGQGTLVTVSS

SEQ ID NO: 96 - DNA, 351 bp, synthetic construct
  misc_feature 1..351: note = Synthesized
  source 1..351: mol_type = other DNA
caggtgcagctggtgcagtccggcgccgaggtgaagaagcccggcgcctccgtgaaggtgtcctgcaaggcctccggctactccttcacctcctactacatccactgggtgcgccaggcccccggccagggcctggagtggatcggctggatctaccccggctccggcaacaccaagtactccgagaagttcaagggccgcgccaccctgaccgccgacacctccacctccaccgcctacatggagctgcgctccctgcgctccgacgacaccgccgtgtacttctgcgcccgcgacggctgctacggcttcgcctactggggccagggcaccctggtgaccgtgtcctca

SEQ ID NO: 97 - protein, 117 aa, synthetic construct
  REGION 1..117: note = Synthesized
QVQLVQSGAEVKKPGASVKVSCKASGYSFTSYYIHWVRQAPGQGLEWIGWIYPGSGNTKYSEKFKGRATLTADTSTSTAYMELRSLRSDDTAVYFCARDGCYGFAYWGQGTLVTVSS

SEQ ID NO: 98 - DNA, 351 bp, synthetic construct
  misc_feature 1..351: note = Synthesized
  source 1..351: mol_type = other DNA
caggtgcagctggtgcagtccggcgccgaggtgaagaagcccggcgcctccgtgaaggtgtcctgcaaggcctccggctacaccttcacctcctactacatccactgggtgcgccaggcccccggccagggcctggagtggatgggctggatctaccccggctccggcaacaccaagtactccgagaagttcaagggccgcgccaccatgaccgccgacacctccacctccaccgcctacatggagctgcgctccctgcgctccgacgacaccgccgtgtactactgcgcccgcgacggctgctacggcttcgcctactggggccagggcaccctggtgaccgtgtcctca

SEQ ID NO: 99 - protein, 117 aa, synthetic construct
  REGION 1..117: note = Synthesized
QVQLVQSGAEVKKPGASVKVSCKASGYTFTSYYIHWVRQAPGQGLEWMGWIYPGSGNTKYSEKFKGRATMTADTSTSTAYMELRSLRSDDTAVYYCARDGCYGFAYWGQGTLVTVSS

SEQ ID NO: 100 - DNA, 351 bp, synthetic construct
  misc_feature 1..351: note = Synthesized
  source 1..351: mol_type = other DNA
caggtgcagctggtgcagtccggcgccgaggtgaagaagcccggcgcctccgtgaaggtgtcctgcaaggcctccggctactccttcacctcctactacatccactgggtgcgccaggcccccggccagggcctggagtggatcggctggatctaccccggctccggcaacaccaagtactccgagaagttcaagggccgcgccaccctgaccgccgacacctccacctccaccgcctacatggagctgcgctccctgcgctccgacgacaccgccgtgtacttctgcgcccgcgacggctcctacggcttcgcctactggggccagggcaccctggtgaccgtgtcctca

SEQ ID NO: 101 - protein, 117 aa, synthetic construct
  REGION 1..117: note = Synthesized
QVQLVQSGAEVKKPGASVKVSCKASGYSFTSYYIHWVRQAPGQGLEWIGWIYPGSGNTKYSEKFKGRATLTADTSTSTAYMELRSLRSDDTAVYFCARDGSYGFAYWGQGTLVTVSS

SEQ ID NO: 102 - DNA, 351 bp, synthetic construct
  misc_feature 1..351: note = Synthesized
  source 1..351: mol_type = other DNA
caggtgcagctggtgcagtccggcgccgaggtgaagaagcccggcgcctccgtgaaggtgtcctgcaaggcctccggctacaccttcacctcctactacatccactgggtgcgccaggcccccggccagggcctggagtggatgggctggatctaccccggctccggcaacaccaagtactccgagaagttcaagggccgcgccaccatgaccgccgacacctccacctccaccgcctacatggagctgcgctccctgcgctccgacgacaccgccgtgtactactgcgcccgcgacggctcctacggcttcgcctactggggccagggcaccctggtgaccgtgtcctca

SEQ ID NO: 103 - protein, 117 aa, synthetic construct
  REGION 1..117: note = Synthesized
QVQLVQSGAEVKKPGASVKVSCKASGYTFTSYYIHWVRQAPGQGLEWMGWIYPGSGNTKYSEKFKGRATMTADTSTSTAYMELRSLRSDDTAVYYCARDGSYGFAYWGQGTLVTVSS

SEQ ID NO: 104 - DNA, 57 bp, synthetic construct; identical to SEQ ID NO: 27
  misc_feature 1..57: note = Synthesized
  source 1..57: mol_type = other DNA

SEQ ID NO: 105 - protein, 19 aa, synthetic construct
  REGION 1..19: note = Synthesized
MEFGLSWLFLVAILKGVQC

SEQ ID NO: 106 - DNA, 57 bp, synthetic construct
  misc_feature 1..57: note = Synthesized
  source 1..57: mol_type = other DNA
atggactggacctggagcatccttttcttggtggcagcagcaacaggtgcccactcc

SEQ ID NO: 107 - protein, 19 aa, synthetic construct
  REGION 1..19: note = Synthesized
MDWTWSILFLVAAATGAHS

SEQ ID NO: 108 - DNA, 333 bp, synthetic construct
  misc_feature 1..333: note = Synthesized
  source 1..333: mol_type = other DNA
gacatccagctgacccagtccccctcctccctgtccgcctccgtgggcgaccgcgtgaccatcacctgcaaggcctcccagtccgtggactacgacggcgactcctacatgaactggtaccagcagaagcccggcaaggcccccaagttcctgatctgcgccgcctccaacctggagtccggcgtgccctcccgcttctccggctccggctccggcaccgacttcaccctgaccatctcctccctgcagcccgaggacgccgccacctactactgccagcagtccaacgaggaccccttcaccttcggccagggcaccaagctggagatcaag

SEQ ID NO: 109 - protein, 111 aa, synthetic construct
  REGION 1..111: note = Synthesized
DIQLTQSPSSLSASVGDRVTITCKASQSVDYDGDSYMNWYQQKPGKAPKFLICAASNLESGVPSRFSGSGSGTDFTLTISSLQPEDAATYYCQQSNEDPFTFGQGTKLEIK

SEQ ID NO: 110 - DNA, 333 bp, synthetic construct
  misc_feature 1..333: note = Synthesized
  source 1..333: mol_type = other DNA
gacatccagctgacccagtccccctcctccctgtccgcctccgtgggcgaccgcgtgaccatcacctgcaaggcctcccagtccgtggactacgacggcgactcctacatgaactggtaccagcagaagcccggcaaggcccccaagttcctgatctacgccgcctccaacctggagtccggcgtgccctcccgcttctccggctccggctccggcaccgacttcaccctgaccatctcctccctgcagcccgaggacgccgccacctactactgccagcagtccaacgaggaccccttcaccttcggccagggcaccaagctggagatcaag

SEQ ID NO: 111 - protein, 111 aa, synthetic construct
  REGION 1..111: note = Synthesized
DIQLTQSPSSLSASVGDRVTITCKASQSVDYDGDSYMNWYQQKPGKAPKFLIYAASNLESGVPSRFSGSGSGTDFTLTISSLQPEDAATYYCQQSNEDPFTFGQGTKLEIK

SEQ ID NO: 112 - DNA, 333 bp, synthetic construct
  misc_feature 1..333: note = Synthesized
  source 1..333: mol_type = other DNA
gacatccagctgacccagtccccctcctccctgtccgcctccgtgggcgaccgcgtgaccatcacctgcaaggcctcccagtccgtggactacgacggcgactcctacatgaactggtaccagcagaagcccggcaaggcccccaagttcctgatctgcgccgcctccaacctggagtccggcgtgccctcccgcttctccggctccggctccggcaccgacttcaccctgaccatctcctccctgcagcccgaggacttcgccacctactactgccagcagtccaacgaggaccccttcaccttcggccagggcaccaagctggagatcaag

SEQ ID NO: 113 - protein, 111 aa, synthetic construct
  REGION 1..111: note = Synthesized
DIQLTQSPSSLSASVGDRVTITCKASQSVDYDGDSYMNWYQQKPGKAPKFLICAASNLESGVPSRFSGSGSGTDFTLTISSLQPEDFATYYCQQSNEDPFTFGQGTKLEIK

SEQ ID NO: 114 - DNA, 333 bp, synthetic construct
  misc_feature 1..333: note = Synthesized
  source 1..333: mol_type = other DNA
gacatccagctgacccagtccccctcctccctgtccgcctccgtgggcgaccgcgtgaccatcacctgcaaggcctcccagtccgtggactacgacggcgactcctacatgaactggtaccagcagaagcccggcaaggcccccaagttcctgatctacgccgcctccaacctggagtccggcgtgccctcccgcttctccggctccggctccggcaccgacttcaccctgaccatctcctccctgcagcccgaggacttcgccacctactactgccagcagtccaacgaggaccccttcaccttcggccagggcaccaagctggagatcaag

SEQ ID NO: 115 - protein, 111 aa, synthetic construct
  REGION 1..111: note = Synthesized
DIQLTQSPSSLSASVGDRVTITCKASQSVDYDGDSYMNWYQQKPGKAPKFLIYAASNLESGVPSRFSGSGSGTDFTLTISSLQPEDFATYYCQQSNEDPFTFGQGTKLEIK

SEQ ID NO: 116 - DNA, 66 bp, synthetic construct; identical to SEQ ID NO: 25
  misc_feature 1..66: note = Synthesized
  source 1..66: mol_type = other DNA

SEQ ID NO: 117 - protein, 117 aa, synthetic construct
  REGION 1..117: note = Synthesized
QVQLVQSGAEVKKPGASVKVSCKASGYTFTSFGISWVRQAPGQGLEWMGWISPYNGDTNYAQNLQGRVTMTTDTSTSTAYMELRSLRSDDTAVYYCARDRGSMSDYWGQGTLVTVSS

SEQ ID NO: 118 - protein, 107 aa, synthetic construct
  REGION 1..107: note = Synthesized
DIQLTQSPSSLSASVGDRVTITCRASQGIRNDLGWYQQKPGKAPKLLIYAASSLQSGVPSRFSGSGSGTDFTLTISSLQPEDFATYYCLQDYNYPPTFGQGTKLEIK

SEQ ID NO: 119 - protein, 117 aa, synthetic construct
  REGION 1..117: note = Synthesized
QVQLVQSGAEVKKPGASVKVSCKASGYSFTSYYIHWVRQAPGQGLEWMGWIYPGSGNTKYSEKFKGRVTMTTDTSTSTAYMELRSLRSDDTAVYYCARDGCYGFAYWGQGTLVTVSS

SEQ ID NO: 120 - protein, 111 aa, synthetic construct
  REGION 1..111: note = Synthesized
DIQLTQSPSSLSASVGDRVTITCKASQSVDYDGDSYMNWYQQKPGKAPKLLIYAASNLESGVPSRFSGSGSGTDFTLTISSLQPEDFATYYCQQSNEDPFTFGQGTKLEIK

SEQ ID NO: 121 - protein, 117 aa, synthetic construct
  REGION 1..117: note = Synthesized
  VARIANT 11: note = Xaa = V or L
  VARIANT 28: note = Xaa = S or T
  VARIANT 38: note = Xaa = R or K
  VARIANT 48: note = Xaa = M or I
  VARIANT 68: note = Xaa = V or A
  VARIANT 70: note = Xaa = M or L
  VARIANT 72: note = Xaa = T or A
  VARIANT 95: note = Xaa = Y or F
  VARIANT 101: note = Xaa = C or S
QVQLVQSGAEXKKPGASVKVSCKASGYXFTSYYIHWVXQAPGQGLEWXGWIYPGSGNTKYSEKFKGRXTXTXDTSTSTAYMELRSLRSDDTAVYXCARDGXYGFAYWGQGTLVTVSS

SEQ ID NO: 122 - protein, 111 aa, synthetic construct
  REGION 1..111: note = Synthesized
  VARIANT 50: note = Xaa = L or F
  VARIANT 53: note = Xaa = Y or C
  VARIANT 87: note = Xaa = F or A
DIQLTQSPSSLSASVGDRVTITCKASQSVDYDGDSYMNWYQQKPGKAPKXLIXAASNLESGVPSRFSGSGSGTDFTLTISSLQPEDXATYYCQQSNEDPFTFGQGTKLEIK

SEQ ID NO: 123 - protein, 8 aa, synthetic construct
  REGION 1..8: note = Synthesized
  VARIANT 3: note = Xaa = C or M or S or T
DGXYGFAY

SEQ ID NO: 124 - protein, 218 aa, synthetic construct
  REGION 1..218: note = Synthesized
DIQLTQSPSSLSASVGDRVTITCKASQSVDYDGDSYMNWYQQKPGKAPKFLICAASNLESGVPSRFSGSGSGTDFTLTISSLQPEDFATYYCQQSNEDPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 125 - protein, 217 aa, synthetic construct
  REGION 1..217: note = Synthesized
DIQLTQSPSSLSASVGDRVTITCKASQSVDYDGDSYMNWYQQKPGKAPKFLICAASNLESGVPSRFSGSGSGTDFTLTISSLQPEDFATYYCQQSNEDPFTFGQGTKLEIKTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 126 - protein, 447 aa, synthetic construct
  REGION 1..447: note = Synthesized
QVQLVQSGAEVKKPGASVKVSCKASGYTFTSYYIHWVRQAPGQGLEWMGWIYPGSGNTKYSEKFKGRATMTADTSTSTAYMELRSLRSDDTAVYYCARDGCYGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNVKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 127 - protein, 447 aa, synthetic construct
  REGION 1..447: note = Synthesized
QVQLVQSGAEVKKPGASVKVSCKASGYTFTSYYIHWVRQAPGQGLEWMGWIYPGSGNTKYSEKFKGRATMTADTSTSTAYMELRSLRSDDTAVYYCARDGCYGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 128 - protein, 326 aa, synthetic construct
  REGION 1..326: note = Synthesized
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 129 - protein, 330 aa, synthetic construct
  REGION 1..330: note = Synthesized
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 130 - protein, 113 aa, synthetic construct
  REGION 1..113: note = Synthesized
EVQLVQSGGGLVEPGGSLRLSCAVSGFDFEKAWMSWVRQAPGQGLQWVARIKSTADGGTTSYAAPVEGRFIISRDDSRNMLYLQMNSLKTEDTAVYYCTSAHWGQGTLVTVSS

SEQ ID NO: 131 - protein, 5 aa, synthetic construct
  REGION 1..5: note = Synthesized
KAWMS

SEQ ID NO: 132 - protein, 19 aa, synthetic construct
  REGION 1..19: note = Synthesized
RIKSTADGGTTSYAAPVEG

SEQ ID NO: 133 - skipped

SEQ ID NO: 134 - protein, 108 aa, synthetic construct
  REGION 1..108: note = Synthesized
QSVLTQPPSVSVSPGQTARITCSGEALPMQFAHWYQQRPGKAPVIVVYKDSERPSGVPERFSGSSSGTTATLTITGVQAEDEADYYCQSPDSTNTYEVFGGGTKLTVL

SEQ ID NO: 135 - protein, 11 aa, synthetic construct
  REGION 1..11: note = Synthesized
SGEALPMQFAH

SEQ ID NO: 136 - protein, 7 aa, synthetic construct
  REGION 1..7: note = Synthesized
KDSERPS

SEQ ID NO: 137 - protein, 11 aa, synthetic construct
  REGION 1..11: note = Synthesized
QSPDSTNTYEV

SEQ ID NO: 138 - protein, 124 aa, synthetic construct
  REGION 1..124: note = Synthesized
EVQLVESGAEVKKPGASVKVSCKASGYTFTNYAMHWVRQAPGQRLEWMGWINAGNGKRKYSQKFQDRVTINRDTSASTIYMELSSLGSEDTAVYYCAREEDHAGSGSYLSMDVWGQGTLVTVSS

SEQ ID NO: 139 - protein, 5 aa, synthetic construct
  REGION 1..5: note = Synthesized
NYAMH

SEQ ID NO: 140 - protein, 17 aa, synthetic construct
  REGION 1..17: note = Synthesized
WINAGNGKRKYSQKFQD

SEQ ID NO: 141 - protein, 15 aa, synthetic construct
  REGION 1..15: note = Synthesized
EEDHAGSGSYLSMDV

SEQ ID NO: 142 - protein, 113 aa, synthetic construct
  REGION 1..113: note = Synthesized
DVVMTQSPDSLAVSLGERATINCKSSQNVLYSSNNKNYLAWYQQKPGHPPKLLIYWASTRESGVPDRFSGSGSGTDFTLTITSLQTEDVAVYYCQQYYSSPLTFGGGTKVEIK

SEQ ID NO: 143 - protein, 17 aa, synthetic construct
  REGION 1..17: note = Synthesized
KSSQNVLYSSNNKNYLA

SEQ ID NO: 144 - protein, 7 aa, synthetic construct
  REGION 1..7: note = Synthesized
WASTRES

SEQ ID NO: 145 - protein, 9 aa, synthetic construct
  REGION 1..9: note = Synthesized
QQYYSSPLT

SEQ ID NO: 146 - protein, 5 aa, synthetic construct
  REGION 1..5: note = Synthesized
NYGMS

SEQ ID NO: 147 - protein, 17 aa, synthetic construct
  REGION 1..17: note = Synthesized
SISSGGGSTYYPDNVKG

SEQ ID NO: 148 - protein, 7 aa, synthetic construct
  REGION 1..7: note = Synthesized
GGAGIDY

SEQ ID NO: 149 - protein, 17 aa, synthetic construct
  REGION 1..17: note = Synthesized
KSIQTLLYSSNQKNYLA

SEQ ID NO: 150 - protein, 7 aa, synthetic construct
  REGION 1..7: note = Synthesized
WASIRKS

SEQ ID NO: 151 - protein, 9 aa, synthetic construct
  REGION 1..9: note = Synthesized
QQYYSYPLT

(19) United States
(12) Patent Application Publication
(10) Pub. No.: US 2026/0234230 A1
(43) Pub. Date: Aug. 13, 2026

(54) DOSING REGIMES FOR TREATMENT OF SYNUCLEINOPATHIES

(71) Applicants: Prothena Biosciences Limited, Dublin 2 (IE); F. Hoffmann-La Roche AG, Basel (CH)

(72) Inventors: Jay Soto, San Francisco, CA (US); Daniel Keith Ness, San Mateo, CA (US); Martin Koller, Rancho Santa Fe, CA (US); Diane Mould, Fort Myers, FL (US); Frank Boess, Basel (CH); Meret Martin-Facklam, Basel (CH); Valerie Cosson, Basel (CH); Hans Peter Grimm, Basel (CH); Ronald Gieschke, Basel (CH); Sara Belli, Basel (CH); Silke Weber, Basel (CH)

(21) Appl. No.: 19/556,813

(22) Filed: Mar. 4, 2026

Related U.S. Application Data
(63) Continuation of application No. 16/936,457, filed on Jul. 23, 2020, now Pat. No. 12,595,295.
Application No. 16/936,457 is a continuation of application No. 16/145,618, filed on Sep. 28, 2018, now abandoned.
(60) Provisional application No. 62/564,992, filed on Sep. 28, 2017.

(51) Int. Cl. C07K 16/18; A61B 5/00; A61B 5/11; A61K 9/00; A61K 31/198; A61K 39/00; A61P 25/16; A61P 25/28; C07K 16/28

(52) U.S. Cl. CPC C07K 16/18; A61B 5/0004; A61B 5/4848; A61K 9/0019; A61K 31/198; A61P 25/16; A61P 25/28; C07K 16/2857; A61B 5/11; A61B 5/1101; A61B 5/4082; A61K 2039/505; A61K 2039/545

(57) ABSTRACT

The invention provides dosage regimes for treatment of synucleinopathies. In one regime, a subject receives 3000-5000 mg of an antibody intravenously every 3-5 weeks. In another regime, a subject receives 1300-1700 mg of an antibody intravenously every 3-5 weeks.

This application is a continuation of U.S. application Ser. No. 16/936,457, filed on Jul. 23, 2020, which is a continuation of U.S. application Ser. No. 16/145,618, filed on Sep. 28, 2018, now abandoned, which claims the benefit of U.S. Provisional Application No. 62/564,992, filed on Sep. 28, 2017. The entire disclosure contents of these applications are herewith incorporated by reference in their entirety into the present application.

### REFERENCE TO AN ELECTRONIC SEQUENCE LISTING

The instant application contains a Sequence Listing that has been submitted electronically and is hereby incorporated by reference in its entirety. The Sequence Listing was created on Mar. 4, 2026, is named “17-1537-US-CON2_SequenceListing.xml”, and is 217,598 bytes in size.

### BACKGROUND

Lewy body diseases (LBDs) are characterized by degeneration of the dopaminergic system, motor alterations, cognitive impairment, and formation of Lewy bodies (LBs) and/or Lewy neurites. (McKeith et al., Neurology (1996) 47:1113-24). Lewy Body disease include Parkinson's disease (including idiopathic Parkinson's disease), and Dementia with Lewy Bodies (DLB). Lewy body diseases are a common cause for movement disorders and cognitive deterioration in the aging population (Galasko et al., Arch. Neurol. (1994) 51:888-95). Constipation is another common symptom of subjects with Lewy body disease (Ondo et al., Neurology 2012; 78; 1650-1654; Ashraf et al., Movement Disorders 12, 946-951 (1997)).

Alpha-synuclein is a protein that is normally associated with synapses and is believed to play a role in neural plasticity, learning and memory. Alpha-synuclein is also implicated with a central role in pathogenesis of Lewy body disease. The protein can aggregate to form insoluble fibrils in pathological conditions. For example, alpha-synuclein accumulates in LBs (Spillantini et al., Nature (1997) 388:839-40; Takeda et al., J. Pathol. (1998) 152:367-72; Wakabayashi et al., Neurosci. Lett. (1997) 239:45-8). Mutations in the alpha-synuclein gene co-segregate with rare familial forms of parkinsonism (Kruger et al., Nature Gen. (1998) 18:106-8; Polymeropoulos, et al., Science (1997) 276:2045-7). Over expression of alpha-synuclein in transgenic mice (Masliah et al., Science (2000) 287:1265-9) and Drosophila (Feany et al., Nature (2000) 404:394-8) mimics several pathological aspects of Lewy body disease. Soluble oligomers of alpha-synuclein may be neurotoxic (Conway et al., Proc Natl Acad Sci USA (2000) 97:571-576. The accumulation of alpha-synuclein with similar morphological and neurological alterations in species and animal models as diverse as humans, mice, and flies suggests that this molecule contributes to the development of Lewy body disease.

Immunotherapy directed against alpha-synuclein has been reported to reduce alpha-synuclein deposits and behavioral symptoms in mouse models of Lewy body disease (Masliah et al., PLOS ONE 6(4): e19338. doi: 10.1371/journal.pone.0019338).

### SUMMARY OF THE CLAIMED INVENTION

In one aspect, the invention provides a method of treating or effecting prophylaxis of a subject having or at risk of a synucleinopathy comprising intravenously administering to the subject a dose of 3000-5000 mg of an antibody against alpha-synuclein at intervals of 3-5 weeks, or administering another regime that delivers the antibody to the subject with substantially the same area under the curve. In some such methods the dose is 3500-4500 mg.

In some such methods, the antibody is administered to a plurality of subjects having or at risk of the synucleinopathy wherein subjects receive either one or two fixed doses. In some such methods, subjects with a weight less than 65 kg receive a dose of 3500 mg antibody and subjects with a weight greater or equal to 65 kg receive a dose of 4500 mg.

In some such methods, the dose of 45-75 mg/kg. In some such methods, the dose is 50-70 mg/kg.

In some such methods, the intervals are 28 days. In some such methods, the subject receives at least six doses of the antibody at the intervals of 3-5 weeks. In some such methods, the subject receives at least 12 doses of the antibody at the intervals of 3-5 weeks. In some such methods, the subject receives at least 18 doses of the antibody at the intervals of 3-5 weeks. In some such methods, the interval is 4 weeks. In some such methods, the subject receives the antibody every 4 weeks for at least 52 weeks.

Some such methods further comprise monitoring the subject for a change in movement, cognitive deficit, autonomic dysfunction, gastrointestinal dysfunction, visual hallucination or a psychological symptom.

In some such methods, the monitoring comprises
(a) providing a subject with a mobile device programmed to receive and transmit data acquired from sensors internal and/or external to the device relating to movement deficits of a subject having or suspected of having a synucleinopathy, whereafter the subject undergoes a series of movements to reveal movement deficits, if present, and the internal or external sensors of the device acquire data relating to the movements;
(b) collecting data transmitted from the mobile device; and
(c) comparing the data acquired from the subjects with control data to assess presence or extent of movement deficits in the subject.

In some such methods, the mobile device is programmed to receive and transmit data from at least two external sensors attached to upper and lower limbs of the subject. In some such methods, the mobile device acquires data from sensors on the upper and lower limbs of the subject. In some such methods, the mobile device is carried by the subject and acquires data from an internal sensor. In some such methods, the series of movements includes tapping the device, sitting and standing. In some such methods, the monitoring indicates a reduced movement deficit or a reduced cognitive deficit responsive to administering the antibody.

In some such methods, the antibody binds within residues 115-130 of alpha synuclein.

In some such methods, the antibody binds with residues 118-126 of alpha synuclein.

In some such methods, the antibody binds within residues 117-123 of alpha synuclein. In some such methods, the antibody is NI-202.21D11. In some such methods, the antibody comprises three heavy CDRs designated SEQ ID NOs:139-141 respectively and three light chain CDRs designated SEQ ID NOs: 143-145 respectively. In some such methods, the antibody comprises a heavy chain variable region designated SEQ ID NO:138 and a light chain variable region designated SEQ ID NO:142.

In some such methods, the antibody comprises the three light chain Kabat CDRs of SEQ ID NO:5 and the three heavy chain Kabat CDRs of SEQ ID NO:10. In some such methods, the antibody comprises a heavy chain variable region of any of SEQ ID NOs:8-11 and a light chain variable region of any of SEQ ID NO:3-5, preferably wherein the heavy chain variable region is of SEQ ID NO:10 and a light chain variable region of SEQ ID NO:5.

In some such methods, the antibody is of human IgG1 isotype. In some such methods, the antibody comprises a heavy chain constant region of SEQ ID NO:35 provided the C-terminal lysine of SEQ ID NO:35 may be absent and a light chain constant region of SEQ ID NO:30. In some such methods, the antibody comprises a heavy chain constant region of SEQ ID NO:35 provided the C-terminal lysine of SEQ ID NO:35 may be absent and a light chain constant region of SEQ ID NO:13.

In some such methods, the antibody is 9E4. In some such methods, the antibody comprises three heavy CDRs designated SEQ ID NOs: 146-148 respectively and three light chain CDRs designated SEQ ID NOs: 149-151 respectively. In some such methods, the antibody comprises a heavy chain designated SEQ ID NO:37 and a light chain designated SEQ ID NO:32, wherein the C-terminal lysine of SEQ ID NO:37 may be absent.

In some such methods, the antibody binds within residues 1-20 of alpha-synuclein. In some such methods, the antibody binds with residues 4-15 of alpha-synuclein.

In some such methods, the antibody is NI-202.12F4. In some such methods, the antibody comprises three heavy CDRs designated SEQ ID NOs:131-133 respectively and three light chain CDRs designated SEQ ID NOs: 135-137 respectively. In some such methods, the antibody comprises a heavy chain variable region designated SEQ ID NO:130 and a light chain variable region designated SEQ ID NO:134.

In some such methods, the subject has a weight less than 65 kg and is administered a dose of 3500 mg antibody every 4 weeks.

In some such methods, the administration of the antibody within the range of 3000-5000 mg is preceded by administering a loading dose of 2000 mg of the antibody and optionally uptitration at one or more subsequent dose at greater or equal to 2000 mg but less than 3500 mg until a dose of 3500 mg is reached, all doses being separated by intervals of 3-5 weeks.

In some such methods, the 3500 mg dose is administered in the next interval after the 2000 mg dose. In some such methods, the dose is gradually increased over several intervals prior to administration of the 3500 mg dose. In some such methods, the subject has a weight greater than 65 kg and is administered a dose of 4500 mg antibody every 4 weeks.

In some such methods, the antibody is administered at 4500 mg preceded by administration of a loading dose of 2000 mg of the antibody and optionally uptitration with one or more subsequent doses of greater than or equal to 2000 mg but less than 4500 mg until a dose of 4500 mg is reached with all doses being separated by intervals of 3-5 weeks.

In some such methods, the 4500 mg dose is administered in the next interval after the 2000 mg dose. In some such methods, the dose is gradually increased over several intervals prior to administration of the 4500 mg dose.

Some such methods further comprise monitoring the subject for a change in movement, cognitive deficit autonomic dysfunction, gastrointestinal dysfunction, visual hallucination or a psychological symptom.

In some such methods, the monitoring comprises
(a) providing a subject with a mobile device programmed to receive and transmit data acquired from sensors internal and/or external to the device relating to movement deficits of a subject having or suspected of having a synucleinopathy, whereafter the subject undergoes a series of movements to reveal movement deficits, if present, and the internal or external sensors of the device acquire data relating to the movements;
(b) collecting data transmitted from the mobile device; and
(c) comparing the data acquired from the subjects with control data to assess presence or extent of movement deficits in the subject.

In some such methods, the mobile device is programmed to receive and transmit data from at least two external sensors attached to upper and lower limbs of the subject. In some such methods, the mobile device acquires data from sensors on the upper and lower limbs of the subject. In some such methods, the mobile device is carried by the subject and acquires data from an internal sensor. In some such methods, the series of movements includes tapping the device, sitting and standing. In some such methods, the monitoring indicates a reduced movement deficit or a reduced cognitive deficit responsive to administering the antibody.

In some such methods, the synucleinopathy is Parkinson's disease. In some such methods, the synucleinopathy is dementia with Lewy bodies. In some such methods, the synucleinopathy is multiple system atrophy. In some such methods, the synucleinopathy is progressive supra nuclear palsy. In some such methods, the synucleinopathy is REM sleep behavior disorder. In some such methods, the synucleinopathy is Alzheimer's disease with amygdala Lewy bodies. In some such methods, the subject has the disease.

In some such methods, the subject is not receiving symptomatic treatment for Parkinson's disease concomitant with the antibody. In some such methods, the antibody is administered concomitantly with levodopa.

In another aspect, the invention provides a method of treating or effecting prophylaxis of a subject having a Lewy body disease comprising intravenously administering to the subject a dose of 1300-1700 mg of an antibody against alpha-synuclein at intervals of 3-5 weeks, or administering another regime that delivers the antibody to the subject with substantially the same area under the curve.

In some such methods, the dose is 1500 mg. In some such methods, the antibody is administered to a plurality of subjects having Lewy body disease wherein subjects receive either one or two fixed doses. In some such methods, the subject receives a dose of 18-25 mg/kg. In some such methods, the subject receives a dose of 20 mg/kg.

In some such methods, the intervals are 28 days. In some such methods, the subject receives at least six doses of the antibody at the intervals of 3-5 weeks. In some such methods, the subject receives at least 12 doses of the antibody at the intervals of 3-5 weeks. In some such methods, the subject receives at least 18 doses of the antibody at the intervals of 3-5 weeks. In some such methods, the interval is 4 weeks. In some such methods, the subject receives the antibody every 4 weeks for at least 52 weeks.

Some such methods further comprise monitoring the subject for a change in movement, cognitive deficit autonomic dysfunction, gastrointestinal dysfunction, visual hallucination or a psychological symptom.

In some such methods, the monitoring comprises
(a) providing a subject with a mobile device programmed to receive and transmit data acquired from sensors internal and/or external to the device relating to movement deficits of a subject having or suspected of having a Lewy body disease, whereafter the subject undergoes a series of movements to reveal movement deficits, if present, and the internal or external sensors of the device acquire data relating to the movements;
(b) collecting data transmitted from the mobile device; and
(c) comparing the data acquired from the subjects with control data to assess presence or extent of movement deficits in the subject.

In some such methods, the mobile device is programmed to receive and transmit data from at least two external sensors attached to upper and lower limbs of the subject.

In some such methods, the mobile device acquires data from sensors on the upper and lower limbs of the subject. In some such methods, the mobile device is carried by the subject and acquires data from an internal sensor. In some such methods, the series of movements includes tapping the device, sitting and standing. In some such methods, the monitoring indicates a reduced movement deficit or a reduced cognitive deficit responsive to administering the antibody.

In some such methods, the antibody binds within residues 115-130 of alpha synuclein.

In some such methods, the antibody binds with residues 118-126 of alpha synuclein.

In some such methods, the antibody binds within residues 117-123 of alpha synuclein.

In some such methods, the antibody is NI-202.21D11. In some such methods, the antibody comprises three heavy CDRs designated SEQ ID NOs:139-141 respectively and three light chain CDRs designated SEQ ID NOs: 143-145 respectively. In some such methods, the antibody comprises a heavy chain variable region designated SEQ ID NO:138 and a light chain variable region designated SEQ ID NO:142.

In some such methods, the antibody comprises the three light chain Kabat CDRs of SEQ ID NO:5 and the three heavy chain Kabat CDRs of SEQ ID NO:10. In some such methods, the antibody comprises a heavy chain variable region of any of SEQ ID NOs:8-11 and a light chain variable region of any of SEQ ID NO:3-5, preferably wherein the heavy chain variable region is of SEQ ID NO:10 and a light chain variable region of SEQ ID NO:5.

In some such methods, the antibody is of human IgG1 isotype.

In some such methods, the antibody comprises a heavy chain constant region of SEQ ID NO:35 provided the C-terminal lysine may be absent and a light chain constant region of SEQ ID NO:30. In some such methods, the antibody comprises a heavy chain constant region of SEQ ID NO:35 provided the C-terminal lysine may be absent and a light chain constant region of SEQ ID NO:13.

In some such methods, the antibody comprises three heavy CDRs designated SEQ ID NOs: 146-148 respectively and three light chain CDRs designated SEQ ID NOs: 149-151 respectively. In some such methods, the antibody comprises a heavy chain designated SEQ ID NO:37 and a light chain designated SEQ ID NO:32, wherein the C-terminal lysine of SEQ ID NO:37 may be absent.

In some such methods, the antibody binds within residues 1-20 of alpha-synuclein. In some such methods, the antibody binds with residues 4-15 of alpha-synuclein.

In some such methods, the antibody is NI-202.12F4. In some such methods, the antibody comprises three heavy CDRs designated SEQ ID NOs:131-133 respectively and three light chain CDRs designated SEQ ID NOs: 135-137 respectively. In some such methods, the antibody comprises a heavy chain variable region designated SEQ ID NO:130 and a light chain variable region designated SEQ ID NO:134.

In some such methods, the synucleinopathy is Parkinson's disease. In some such methods, the synucleinopathy is dementia with Lewy bodies. In some such methods, the synucleinopathy is multiple system atrophy. In some such methods, the synucleinopathy is progressive supra nuclear palsy. In some such methods, the synucleinopathy is REM sleep behavior disorder. In some such methods, the synucleinopathy is Alzheimer's disease with amygdala Lewy bodies. In some such methods, the subject has the disease.

In some such methods, the subject is not receiving symptomatic treatment for Parkinson's disease concomitant with the antibody. In some such methods, the antibody is administered concomitantly with levodopa.

### Definitions

The term “antibody” includes intact antibodies and binding fragments thereof. Typically, fragments compete with the intact antibody from which they were derived for specific binding to the target. Fragments include separate heavy chains, separate light chains, Fab, Fab′, F(ab′)2, F(ab)c, Fv, single chain antibodies, and single domain antibodies. The term “antibody” also includes a bispecific antibody. A bispecific or bifunctional antibody is an artificial hybrid antibody having two different heavy/light chain pairs and two different binding sites (see, e.g., Songsivilai and Lachmann, Clin. Exp. Immunol., 79:315-321 (1990); Kostelny et al., J. Immunol., 148:1547-53 (1992)).

The basic antibody structural unit is a tetramer of subunits. Each tetramer includes two identical pairs of polypeptide chains, each pair having one “light” chain (about 25 kDa) and one “heavy” chain (about 50-70 kDa). The amino-terminal portion of each chain includes a variable region of about 100 to 110 or more amino acids primarily responsible for antigen recognition. When initially expressed, this variable region is typically linked to a cleavable signal peptide. The variable region without the signal peptide is sometimes referred to as a mature variable region. Thus, for example, a light chain mature variable region means a light chain variable region without the light chain signal peptide. The carboxy-terminal portion of each chain defines a constant region primarily responsible for effector function. A constant region can include any or all of a CH1 region, hinge region, CH2 region, and CH3 region.

Light chains are classified as either kappa or lambda. Heavy chains are classified as gamma, mu, alpha, delta, or epsilon, and define the antibody's isotype as IgG, IgM, IgA, IgD and IgE, respectively. Within light and heavy chains, the variable and constant regions are joined by a “J” region of about 12 or more amino acids, with the heavy chain also including a “D” region of about 10 or more amino acids. (See generally, Fundamental Immunology (Paul, W., ed., 2nd ed. Raven Press, N.Y., 1989), Ch. 7) (incorporated by reference in its entirety for all purposes).

The mature variable regions of each light/heavy chain pair form the antibody binding site. Thus, an intact antibody has two binding sites. Except for bifunctional or bispecific antibodies, the two binding sites are the same. The chains all exhibit the same general structure of relatively conserved framework regions (FR) joined by three hypervariable regions, also called complementarity determining regions (CDRs). The CDRs from the two chains of each pair are aligned by the framework regions, enabling binding to a specific epitope. From N-terminal to C-terminal, both light and heavy chains comprise the regions FR1, CDR1, FR2, CDR2, FR3, CDR3 and FR4. The assignment of amino acids to each region is in accordance with the definitions of Kabat, Sequences of Proteins of Immunological Interest (National Institutes of Health, Bethesda, MD, 1987 and 1991), or Chothia & Lesk, J. Mol. Biol. 196:901-917 (1987); Chothia et al., Nature 342:878-883 (1989). Kabat also provides a widely used numbering convention (Kabat numbering) in which corresponding residues between different heavy chains or between different light chains are assigned the same number.

Percentage sequence identities are determined with antibody sequences maximally aligned by the Kabat numbering convention. After alignment, if a subject antibody region (e.g., the entire mature variable region of a heavy or light chain) is being compared with the same region of a reference antibody, the percentage sequence identity between the subject and reference antibody regions is the number of positions occupied by the same amino acid in both the subject and reference antibody region divided by the total number of aligned positions of the two regions (with gaps not counted) multiplied by 100 to convert to percentage.

For purposes of classifying amino acids substitutions as conservative or non-conservative, amino acids are grouped as follows: Group I (hydrophobic sidechains): Norleucine, Met, Ala, Val, Leu, Ile; Group II (neutral hydrophilic side chains): Cys, Ser, Thr; Group III (acidic side chains): Asp, Glu; Group IV (basic side chains): Asn, Gln, His, Lys, Arg; Group V (residues influencing chain orientation): Gly, Pro; and Group VI (aromatic side chains): Trp, Tyr, Phe. Conservative substitutions involve substitutions between amino acids in the same class. Non-conservative substitutions constitute exchanging a member of one of these classes for a member of another.

Antibodies of the invention typically bind to their designated target with an affinity constant of at least 10⁶, 10⁷, 10⁸, 10⁹, or 10¹⁰ M⁻¹. Such binding is specific binding in that it is detectably higher in magnitude and distinguishable from non-specific binding occurring to at least one unrelated target. Specific binding can be the result of formation of bonds between particular functional groups or particular spatial fit (e.g., lock and key type) whereas nonspecific binding is usually the result of van der Waals forces. Specific binding does not, however, necessarily imply that a monoclonal antibody binds one and only one target.

The term “symptom” refers to subjective evidence of a disease, such as altered gait, as perceived by a subject. A “sign” refers to objective evidence of a disease as observed by a physician.

An individual is at increased risk of a disease if the subject has at least one known risk-factor (e.g., genetic, biochemical, family history, situational exposure) placing individuals with that risk factor at a statistically significant greater risk of developing the disease than individuals without the risk factor. Statistical significance means p≤0.05.

Unless otherwise apparent from the context, the term “about” encompasses values within the standard deviation of the mean of a stated value or +/−5% of a stated value, whichever is greater.

The term “9E4 antibody” refers to any antibody in which each of the CDRs is substantially that of 9E4, and thus includes murine, chimeric, veneered, and humanized 9E4. References to other antibodies, such as 5C1 and 1H7 have corresponding meanings.

Antibodies of the invention can be administered concomitant with another treatment for the same indication as the antibody, meaning that the other treatment is administered at least once during the period in which the antibody is administered, such period beginning one month before the first dosing and ending one month after the last dosing of the antibody. The other treatment can be administered at recurring intervals during this period, which may or may not be the same as the intervals at which the antibody is administered. The other treatment may be a symptomatic treatment.

A treatment is symptomatic if it only affects one or more symptoms of a disease, not its cause, i.e., its etiology.

The term “mobile device” as used herein refers to any portable device which comprises a sensor and data-recording equipment suitable for obtaining the dataset of activity measurements. Typically, the mobile device comprises a sensor for measuring the activity. This may also require a data processor and storage unit as well as a display for electronically simulating an activity test on the mobile device. Moreover, from the activity of the subject data shall be recorded and compiled to a dataset which is to be evaluated by the method of the present invention either on the mobile device itself or on a second device. Depending on the specific setup envisaged, it may be necessary that the mobile device comprises data transmission equipment in order to transfer the acquired dataset from the mobile device to one or more further devices. Particular well suited as mobile devices according to the present invention are smartphones, smartwatches, wearable sensors, portable multimedia devices or tablet computers. Alternatively, portable sensors with data recording and, optionally, processing equipment may be used. Further, depending on the kind of activity test to be performed, the mobile device shall be adapted to display instructions for the subject regarding the activity to be carried out for the test.

Unless otherwise apparent from the context, reference to a range includes any integer within the range.

A treatment regime refers to a combination of parameters characterizing administration of an immunotherapeutic agent including any or all of dose, frequency of administration, route of administration, and total duration of administration.

### BRIEF DESCRIPTION OF THE SEQUENCES

SEQ ID NO:1 is an m9E4VL variable region.

SEQ ID NO:2 is an 63102889Hu9E4VLFr variable region.

SEQ ID NO:3 is an Hu9E4VLv1 variable region.

SEQ ID NO:4 is an Hu9E4VLv2 (No backmutation) variable region.

SEQ ID NO:5 is an Hu9E4VLv3 variable region.

SEQ ID NO:6 is an m9E4VH variable region.

SEQ ID NO:7 is an 1791009Hu9E4VHFr variable region.

SEQ ID NO:8 is an Hu9E4VHv1 variable region.

SEQ ID NO:9 is an Hu9E4VHv2 variable region.

SEQ ID NO:10 is an Hu9E4VHv3 variable region.

SEQ ID NO:11 is an Hu9E4VHv4 (no backmutation) variable region.

SEQ ID NO:12 is the amino acid sequence of natural human wildtype alpha-synuclein.

SEQ ID NO:13 is a humanized 9E4 light chain constant region (with R) (common for v1, v2, v3).

SEQ ID NO:14 is a humanized 9E4 light chain constant region (with R) nucleotide sequence (common for v1, v2, v3).

SEQ ID NO:15 is an amino acid sequence of an exemplary human IgG1 constant region.

SEQ ID NO:16 is a nucleic acid sequence encoding an exemplary human IgG1 constant region.

SEQ ID NO:17 is an Hu9E4VLv1 nucleotide sequence variable region.

SEQ ID NO:18 is an Hu9E4VLv2 nucleotide sequence (no backmutation) variable region.

SEQ ID NO:19 is an Hu9E4VLv3 nucleotide sequence variable region.

SEQ ID NO:20 is an Hu9E4VHv1 nucleotide sequence variable region.

SEQ ID NO:21 is an Hu9E4VHv2 nucleotide sequence variable region.

SEQ ID NO:22 is an Hu9E4VHv3 nucleotide sequence variable region.

SEQ ID NO:23 is an Hu9E4VHv4 nucleotide sequence (no backmutation) variable region.

SEQ ID NO:24 is an Hu9E4VL signal peptide (common for v1, v2, v3).

SEQ ID NO:25 is an Hu9E4VL signal peptide nucleotide sequence (common for v1, v2, v3).

SEQ ID NO:26 is an Hu9E4VH signal peptide (common for v1, v2, v3).

SEQ ID NO:27 is an Hu9E4VH signal peptide nucleotide sequence (common for v1, v2, v3, v4).

SEQ ID NO:28 is an Hu9E4VL alternative.

SEQ ID NO:29 is an Hu9E4VH alternative.

SEQ ID NO:30 is an amino acid sequence of an exemplary human kappa light chain constant region.

SEQ ID NO:31 is a humanized 9E4 light chain constant region (without R) nucleotide sequence (common for v1, v2, v3).

SEQ ID NO:32 is a humanized 9E4 light chain version 3 (variable region+constant region with Arginine).

SEQ ID NO:33 is a humanized 9E4 light chain version 3 (variable region+constant region without Arginine).

SEQ ID NO:34 is a humanized 9E4 heavy chain version 3 (variable region+constant region).

SEQ ID NO:35 is a humanized 9E4 heavy chain constant region (G1m3 allotype; BIP version).

SEQ ID NO:36 is a humanized 9E4 heavy chain version 3 (variable region+constant region).

SEQ ID NO:37 is a humanized 9E4 heavy chain version 3 (variable region+alternative constant region G1m3 allotype).

SEQ ID NO:38 is an m5C1 antibody mature heavy chain variable region nucleotide sequence.

SEQ ID NO:39 is an m5C1 antibody heavy chain variable region amino acid sequence.

SEQ ID NO:40 is an m5C1 antibody heavy chain signal peptide nucleotide sequence.

SEQ ID NO:41 is an m5C1 antibody heavy chain signal peptide amino acid sequence.

SEQ ID NO:42 is an m5C1 antibody mature light chain variable region nucleotide sequence.

SEQ ID NO:43 is an m5C1 antibody light chain variable region amino acid sequence.

SEQ ID NO:44 is an m5C1 antibody light chain signal peptide nucleotide sequence.

SEQ ID NO:45 is an m5C1 antibody light chain signal peptide amino acid sequence.

SEQ ID NO:46 is a 5C1 immunogen.

SEQ ID NO:47 is an m5C1 heavy chain CDR1.

SEQ ID NO:48 is an m5C1 heavy chain CDR2.

SEQ ID NO:49 is an m5C1 heavy chain CDR3.

SEQ ID NO:50 is an m5C1 light chain CDR1.

SEQ ID NO:51 is an m5C1 light chain CDR2.

SEQ ID NO:52 is an m5C1 light chain CDR3.

SEQ ID NO:53 is a murine 5C1 heavy chain variable region nucleotide sequence with sequence encoding signal peptide.

SEQ ID NO:54 is a murine 5C1 heavy chain variable region sequence with signal peptide.

SEQ ID NO:55 is a murine 5C1 light chain variable region nucleotide sequence with sequence encoding signal peptide.

SEQ ID NO:56 is a murine 5C1 light chain variable region sequence with signal peptide.

SEQ ID NO:57 is a human VH Acceptor FR (Acc #AAY42876.1).

SEQ ID NO:58 is a humanized 5C1H1.

SEQ ID NO:59 is a humanized 5C1H2.

SEQ ID NO:60 is a humanized 5C1H3.

SEQ ID NO:61 is a humanized 5C1H4.

SEQ ID NO:62 is a humanized 5C1H5.

SEQ ID NO:63 is a nucleic acid sequence encoding humanized 5C1 H1.

SEQ ID NO:64 is a nucleic acid sequence encoding humanized 5C1 H2.

SEQ ID NO:65 is a nucleic acid sequence encoding humanized 5C1H3.

SEQ ID NO:66 is a nucleic acid sequence encoding humanized 5C1 H4.

SEQ ID NO:67 is a nucleic acid sequence encoding humanized 5C1H5.

SEQ ID NO:68 is a human VL Acceptor FR (Acc #CAB51293.1).

SEQ ID NO:69 is a humanized 5C1L1.

SEQ ID NO:70 is a humanized 5C1L2.

SEQ ID NO:71 is a humanized 5C1L3.

SEQ ID NO:72 is a humanized 5C1L4.

SEQ ID NO:73 is a nucleic acid sequence encoding humanized 5C1 L1.

SEQ ID NO:74 is a nucleic acid sequence encoding humanized 5C1 L2.

SEQ ID NO:75 is a nucleic acid sequence encoding humanized 5C1L3.

SEQ ID NO:76 is a nucleic acid sequence encoding humanized 5C1 L4.

SEQ ID NO:77 is a nucleic acid sequence encoding an exemplary human kappa light chain constant region.

SEQ ID NO:78 is a non-amyloid component (NAC) domain of alpha-synuclein as reported by Jensen et al.

SEQ ID NO:79 is a non-amyloid component (NAC) domain of alpha-synuclein as reported by Uéda et al.

SEQ ID NO:80 is an m1H7 antibody heavy chain variable nucleotide sequence.

SEQ ID NO:81 is an m1H7 antibody heavy chain variable amino acid sequence.

SEQ ID NO:82 is an m1H7 antibody light chain variable nucleotide sequence.

SEQ ID NO:83 is an m1H7 antibody light chain variable amino acid sequence.

SEQ ID NO:84 is a mature m1H7 antibody heavy chain variable nucleotide sequence.

SEQ ID NO:85 is a mature m1H7 antibody heavy chain variable amino acid sequence.

SEQ ID NO:86 is a mature m1H7 antibody light chain variable nucleotide sequence.

SEQ ID NO:87 is a mature m1H7 antibody light chain variable amino acid sequence.

SEQ ID NO:88 is an m1H7 antibody heavy chain CDR1 (Kabat definition).

SEQ ID NO:89 is an m1H7 antibody heavy chain CDR2 (Kabat definition).

SEQ ID NO:90 is an m1H7 antibody heavy chain CDR3 (Kabat definition).

SEQ ID NO:91 is an m1H7 antibody light chain CDR1 (Kabat definition).

SEQ ID NO:92 is an m1H7 antibody light chain CDR2 (Kabat definition).

SEQ ID NO:93 is an m1H7 antibody light chain CDR3 (Kabat definition).

SEQ ID NO:94 is an Hu1H7VHv1 nucleotide sequence.

SEQ ID NO:95 is an Hu1H7VHv1 amino acid sequence.

SEQ ID NO:96 is an Hu1H7VHv2 nucleotide sequence.

SEQ ID NO:97 is an Hu1H7VHv2 amino acid sequence.

SEQ ID NO:98 is an Hu1H7VHv3 nucleotide sequence.

SEQ ID NO:99 is an Hu1H7VHv3 amino acid sequence.

SEQ ID NO:100 is an Hu1H7VHv4 nucleotide sequence.

SEQ ID NO:101 is an Hu1H7VHv4 amino acid sequence.

SEQ ID NO:102 is an Hu1H7VHv5 nucleotide sequence.

SEQ ID NO:103 is an Hu1H7VHv5 amino acid sequence.

SEQ ID NO:104 is an Hu1H7VH signal peptide nucleotide sequence.

SEQ ID NO:105 is an Hu1H7VH signal peptide amino acid sequence.

SEQ ID NO:106 is an Hu1H7VH signal peptide nucleotide sequence.

SEQ ID NO:107 is an Hu1H7VH signal peptide amino acid sequence.

SEQ ID NO:108 is an Hu1H7VLv1 nucleotide sequence.

SEQ ID NO:109 is an Hu1H7VLv1 amino acid sequence.

SEQ ID NO:110 is an Hu1H7VLv2 nucleotide sequence.

SEQ ID NO:111 is an Hu1H7VLv2 amino acid sequence.

SEQ ID NO:112 is an Hu1H7VLv3 nucleotide sequence.

SEQ ID NO:113 is an Hu1H7VLv3 amino acid sequence.

SEQ ID NO:114 is an Hu1H7VLv4 nucleotide sequence.

SEQ ID NO:115 is an Hu1H7VLv4 amino acid sequence.

SEQ ID NO:116 is an Hu1H7VL signal peptide nucleotide sequence.

SEQ ID NO:117 is an amino acid sequence of BAC02037 (GI-21670055) human acceptor used for heavy chain framework.

SEQ ID NO:118 is an amino acid sequence of AAY33358 GI-63102905) human acceptor used for light chain framework.

SEQ ID NO:119 is an Hu1H7VH with no backmutation or CDR mutation.

SEQ ID NO:120 is an Hu1H7VL with no backmutation or CDR mutation.

SEQ ID NO:121 is an Hu1H7VH alternative.

SEQ ID NO:122 is an Hu1H7VL alternative.

SEQ ID NO:123 is an Hu1H7VH CDR3 alternative.

SEQ ID NO:124 is a humanized 1H7 light chain version 3 (variable region+constant region with Arginine).

SEQ ID NO:125 is a humanized 1H7 light chain version 3 (variable region+constant region without Arginine).

SEQ ID NO:126 is a humanized 1H7 heavy chain version 3 (variable region+constant region).

SEQ ID NO:127 is a humanized 1H7 heavy chain version 3 (variable region+constant region G1m3 allotype).

SEQ ID NO:128 is a humanized 1H7 heavy chain constant region (IgG2).

SEQ ID NO:129 is a humanized 1H7 heavy chain constant region (G1m1 allotype).

SEQ ID NO:130 is a NI-202.12F4-VHA1b variable heavy chain (VH) sequence.

SEQ ID NO:131 is a NI-202.12F4-VHA1b CDR1 sequence.

SEQ ID NO:132 is a NI-202.12F4-VHA1b CDR2 sequence

SEQ ID NO:133 is a NI-202.12F4-VHA1b CDR3 sequence.

SEQ ID NO:134 is a NI-202.12F4-VLa1 variable light chain (VL) sequence.

SEQ ID NO:135 is a NI-202.12F4-VLa1 CDR1 sequence.

SEQ ID NO:136 is a NI-202.12F4-VLa1 CDR2 sequence.

SEQ ID NO:137 is a NI-202.12F4-VLa1 CDR3 sequence.

SEQ ID NO:138 is a NI-202.21D11-VH sequence.

SEQ ID NO:139 is a NI-202.21D11-VH CDR1 sequence.

SEQ ID NO:140 is a NI-202.21D11-VH CDR2 sequence.

SEQ ID NO:141 is a NI-202.21D11-VH CDR3 sequence.

SEQ ID NO:142 is a NI-202.21D11-VK sequence.

SEQ ID NO:143 is a NI-202.21D11 VK CDR1 sequence.

SEQ ID NO:144 is a NI-202.21D11 VK CDR2 sequence.

SEQ ID NO:145 is a NI-202.21D11 VK CDR3 sequence.

SEQ ID NO:146 is a 9E4 VH CDR1 sequence.

SEQ ID NO:147 is a 9E4 VH CDR2 sequence.

SEQ ID NO:148 is a 9E4 VH CDR3 sequence.

SEQ ID NO:149 is a 9E4 VL CDR1 sequence.

SEQ ID NO:150 is a 9E4 VL CDR2 sequence.

SEQ ID NO:151 is a 9E4 VL CDR3 sequence.

### DETAILED DESCRIPTION

### I. General

The invention provides dosing regimes for immunotherapy of synucleinopathies. Subjects receive an antibody against alpha-synuclein at a dose of 3000-5000 mg or 1300-1700 mg intravenously every 3-5 weeks. Such doses can be preceded by one or more lower loading doses.

### II. Target Molecule

Natural human wild type alpha-synuclein is a peptide of 140 amino acids having the following amino acid sequence:

(SEQ ID NO: 12)
MDVFMKGLSK AKEGVVAAAE KTKQGVAEAA GKTKEGVLYV
GSKTKEGVVH GVATVAEKTK EQVTNVGGAV VTGVTAVAQK
TVEGAGSIAA ATGFVKKDQL GKNEEGAPQE GILEDMPVDP
DNEAYEMPSE EGYQDYEPEA

(Ueda et al., Proc. Natl. Acad. Sci. USA (1993) 90:11282-6); GenBank accession number: P37840. The protein has three recognized domains: an-N-terminal KTKE repeat domain covering amino acids 1-61; a NAC (Non-amyloid component) domain running from about amino acids 60-95; and a C-terminal acidic domain running from about amino acid 98 to 140.

Unless otherwise apparent from the context, reference to alpha-synuclein or its fragments includes the natural human wildtype amino acid sequences indicated above, and human allelic variants thereof, particularly those associated with Lewy body disease (e.g., variants E46K, A30P and A53T, with the first letter indicating the amino acid in SEQ ID NO:12, the number indicating the codon position in SEQ ID NO:12, and the second letter indicating the amino acid in the allelic variant). Such variants can optionally be present individually or in any combination in any of the aspects of the invention described below.

### III. Synucleinopathies

Synucleinopathies are characterized by excessive accumulation of alpha-synuclein, which may or may not involve the formation of Lewy bodies (LBs). Lewy Body Diseases (LBD), for example, are characterized by degeneration of the dopaminergic system, motor alterations, cognitive impairment, and formation of Lewy bodies. (McKeith et al., Neurology (1996) 47:1113-24). Lewy Bodies are spherical protein deposits found in nerve cells. Their presence in the brain disrupts the brain's normal function interrupting the action of chemical messengers including acetylcholine and dopamine. Synucleinopathies include Parkinson's disease (including idiopathic Parkinson's disease), Dementia with Lewy Bodies (DLB) (also known as Diffuse Lewy Body Disease (DLBD)), Lewy Body variant of Alzheimer's disease (LBV), Combined Alzheimer's and Parkinson disease, progressive supra nuclear palsy (PSP), and multiple system atrophy (MSA; e.g., Olivopontocerebellar Atrophy, Striatonigral Degeneration, and Shy-Drager Syndrome). DLB shares symptoms of both Alzheimer's and Parkinson's disease. DLB differs from Parkinson's disease mainly in the location of Lewy Bodies. In DLB Lewy Bodies form mainly in the cortex. In Parkinson's disease, they form mainly in the substantia nigra. Other synucleinopathies include Pure Autonomic Failure, Lewy Body dysphagia, Incidental LBD, and Inherited LBD (e.g., mutations of the alpha-synuclein gene, PARK3 and PARK4).

### IV. Immunotherapy Agents

Passive immunotherapy involves treatment with an antibody specifically binding to alpha-synuclein.

### 1. Antibodies

### A. Binding Specificity and Functional Properties

The present methods can include antibodies specifically binding to an epitope within residues 1-15, 4-15, 1-20, 91-99, 115-130, 117-123 or 118-126 of human alpha-synuclein. Antibodies can be monoclonal or polyclonal. A polyclonal serum can be specific to an epitope or range of amino acids within alpha-synuclein in that it lacks antibodies binding outside the epitope or range. Antibodies can be non-human, chimeric, veneered, humanized, or human among other possibilities.

Some antibodies are bispecific, one arm of which is an antibody against alpha-synculein and the other arm of which is an antibody that binds to a receptor expressed on the blood brain barrier, such as an insulin receptor, an insulin-like growth factor (IGF) receptor, a leptin receptor, or a lipoprotein receptor, or preferably a transferrin receptor (Friden et al., PNAS 88:4771-4775, 1991; Friden et al., Science 259:373-377, 1993). Such a bispecific antibody can be transferred cross the blood brain barrier by receptor-mediated transcytosis. Brain uptake of the bispecific antibody can be further enhanced by engineering the bi-specific antibody to reduce its affinity to the blood brain barrier receptor. Reduced affinity for the receptor resulted in a broader distributioin in the brain (see, e.g., Atwal. et al. Sci. Trans. Med. 3, 84ra43, 2011; Yu et al. Sci. Trans. Med. 3, 84ra44, 2011).

Exemplary bispecific antibodies can also be (1) a dual-variable-domain antibody (DVD-Ig), where each light chain and heavy chain contains two variable domains in tandem through a short peptide linkage (Wu et al., Generation and Characterization of a Dual Variable Domain Immunoglobulin (DVD-Ig™) Molecule, In: Antibody Engineering, Springer Berlin Heidelberg (2010)); (2) a Tandab, which is a fusion of two single chain diabodies resulting in a tetravalent bispecific antibody that has two binding sites for each of the target antigens; (3) a flexibody, which is a combination of scFvs with a diabody resulting in a multivalent molecule; (4) a so called “dock and lock” molecule, based on the “dimerization and docking domain” in Protein Kinase A, which, when applied to Fabs, can yield a trivalent bispecific binding protein consisting of two identical Fab fragments linked to a different Fab fragment; (5) a so-called Scorpion molecule, comprising, e.g., two scFvs fused to both termini of a human Fc-region. Examples of platforms useful for preparing bispecific antibodies include BiTE (Micromet), DART (MacroGenics), Fcab and Mab2 (F-star), Fc-engineered IgGl (Xencor) or DuoBody (based on Fab arm exchange, Genmab).

Another exemplary antibody is mAb 9E4, which binds to an epitope within residues 118-126 of human alpha-synuclein. The cell line designated JH17.9E4.3.37.1.14.2 producing the antibody 9E4 has the ATCC accession number PTA-8221 having been deposited under the provisions of the Budapest Treaty with the American Type Culture Collection (ATCC, P.O. Box 1549, Manassas, Va. 20108) on Feb. 26, 2007. The murine light and heavy chain variable sequences are SEQ ID NO:1 and SEQ ID NO:6, respectively. Murine mAb 9E4 comprises a VH-CDR1 of SEQ ID NO:146, a VH-CDR2 of SEQ ID NO:147, a VH-CDR3 of SEQ ID NO:148, a VL-CDR1 of SEQ ID NO:149, a VL-CDR2 of SEQ ID NO:150, and a VL-CDR3 of SEQ ID NO:151.

Another exemplary antibody is mAb 5C1. The murine light and heavy chain variable sequences are SEQ ID NO:43 and SEQ ID NO:39, respectively.

Another exemplary antibody is mAb 1H7. The cell line designated JH17.1H7.4.24.34 producing the antibody 1H7 has the ATCC accession number PTA-8220 having been deposited under the provisions of the Budapest Treaty with the American Type Culture Collection (ATCC, P.O. Box 1549, Manassas, Va. 20108) on Feb. 26, 2007. The murine light and heavy chain variable sequences are SEQ ID NO:87 and SEQ ID NO:85, respectively.

Such antibodies are described in WO 06/020581 and U.S. Application Nos. 61/591,835, 61/711,207, 13/750,983, 61/711,204, 61/719,281, 61/840,432, 61/872,366, 14/049,169, 61/553,131, 61/711,204, 61/843,011, and Ser. No. 13/662,261, all of which are incorporated herein by reference for all purposes.

Another exemplary antibody is NI-202.12F4, which is a human antibody reported to bind to an epitope within residues 4-15 of alpha-synuclein with residue 10 being a major contributor to the epitope (see U.S. Pat. No. 8,940,276; WO2012177972, U.S. Pat. No. 9,580,493). NI-202.12F4 comprises a variable heavy chain (VHA1b) sequence of SEQ ID NO:130 and a variable light chain (VLa1) sequence of SEQ ID NO:134. NI-202.12F4 comprises a VHA1b-CDR1 of SEQ ID NO:131, a VHA1b-CDR2 of SEQ ID NO:132, a VHA1b-CDR3 sequence of SEQ ID NO:133 (Ala-His), a VLa1-CDR1 sequence of SEQ ID NO:135, a VLa1-CDR2 sequence of SEQ ID NO: 136, and VLa1-CDR3 sequence of SEQ ID NO:137. Another exemplary antibody is NI-202.21D11, which has been reported to bind to an epitope within residues 117-123 of alpha-synuclein with residues 121 and 122 contributing most to the epitope (see U.S. Pat. No. 9,580,493). NI-202.21D11 comprises a variable heavy chain (VH) sequence of SEQ ID NO:138 and a variable light chain (VK) sequence of SEQ ID NO:142. NI-202.21D11 comprises a VH-CDR1 of SEQ ID NO:139, a VH-CDR2 of SEQ ID NO:140, a VH-CDR3 sequence of SEQ ID NO:141, a VK-CDR1 sequence of SEQ ID NO:143, a VK-CDR2 sequence of SEQ ID NO:144, and VK-CDR3 sequence of SEQ ID NO:145.

### B. Humanized Antibodies

A humanized antibody is a genetically engineered antibody in which the CDRs from a non-human “donor” antibody are grafted into human “acceptor” antibody sequences (see, e.g., Queen et al., U.S. Pat. Nos. 5,530,101 and 5,585,089; Winter et al., U.S. Pat. No. 5,225,539, Carter, U.S. Pat. No. 6,407,213, Adair, U.S. Pat. No. 5,859,205 6,881,557, Foote, U.S. Pat. No. 6,881,557). The acceptor antibody sequences can be, for example, a mature human antibody variable region sequence, a composite of such sequences, a consensus sequence of human antibody sequences (e.g., light and heavy chain variable region consensus sequences of Kabat, 1991, supra), or a germline variable region sequence. Thus, a humanized antibody of the invention includes antibodies having three light chain and three heavy chain CDRs as defined by Kabat entirely or substantially from a murine antibody, such as 9E4, 5C1 or 1H7 (donor antibody) and mature variable region framework sequences and constant regions, if present, entirely or substantially from human antibody sequences. Likewise a humanized heavy chain includes heavy chains having three heavy chain CDRs as defined by Kabat from the heavy chain of the donor antibody, and a mature heavy chain variable sequence and heavy chain constant region sequence, if present, entirely or substantially from human antibody heavy chain sequences. Likewise a humanized light chain includes light chains having three light chain CDRs as defined by Kabat from the light chain of the donor antibody, and a mature light chain variable sequence and light chain constant region sequence, if present, entirely or substantially from human antibody light chain sequences. The mature variable region framework sequences of an antibody chain or the constant region sequence of an antibody chain are substantially from a human mature variable region framework sequence or human constant region sequence, respectively, when at least 85%, 90%, 95%, 96%, 97%, 98%, 99% or 100% of corresponding residues defined by Kabat are identical. A CDR is substantially from a corresponding donor CDR if showing at least 85% identity thereto, except in the case of CDRH2 wherein at least 65% identify is required.

Certain amino acids from the human mature variable region framework residues can be selected for substitution based on their possible influence on CDR conformation and/or binding to antigen. Investigation of such possible influences is by modeling, examination of the characteristics of the amino acids at particular locations, or empirical observation of the effects of substitution or mutagenesis of particular amino acids.

For example, when an amino acid differs between a murine mature variable region framework residue and a selected human mature variable region framework residue, the human framework amino acid can be substituted by the equivalent framework amino acid from the mouse antibody when it is reasonably expected that the amino acid:
(1) noncovalently binds antigen directly,
(2) is adjacent to a CDR region,
(3) otherwise interacts with a CDR region (e.g. is within about 6 Å of a CDR region)
(4) mediates interaction between the heavy and light chains.

Exemplary humanized forms of the mouse 9E4 antibody including three exemplified humanized light chain mature variable regions (Hu9E4VLv1-v3; SEQ ID NOs:3-5) and four exemplified humanized heavy chain mature variable regions (Hu9E4VHv1-v4; SEQ ID NOs: 8-11).

The exemplary light and heavy chain mature variable regions can be paired in any combination. An exemplary combination is Hu9E4VLv3 (SEQ ID NO:5) and Hu9E4VHv3 (SEQ ID NO:10). An exemplary heavy chain constant region is SEQ ID NO:35, with or without the C-terminal lysine. This constant region can be linked to any of the heavy chain variable regions. An exemplary light chain constant region is SEQ ID NO:30. This constant region can be linked to any of the light chain variable regions. An exemplary full length heavy chain has the amino acid sequence of SEQ ID NO:37, with or without the C-terminal lysine. An exemplary full length light chain has the amino acid sequence of SEQ ID NO:32. An exemplary combination is an antibody comprising SEQ ID NO:37 (with or without the C-terminal lysine) and SEQ ID NO:32. The nucleic acids encoding the various heavy and light chains just described are provided in SEQ ID NOs: 17-23. An exemplified humanized antibody has heavy and light chains characterized by SEQ ID NOs: 37 (with or without the C-terminal lysine) and 32.

Exemplary humanized forms of the mouse 5C1 antibody include four exemplified humanized light chain mature variable regions (5C1L1-L4; SEQ ID NOs: 69-72, and five exemplified humanized heavy chain mature variable regions (5C1H1-H4; SEQ ID NOs:58-62). The exemplary light and heavy chain mature variable regions can be paired in any combination. An exemplary heavy chain constant region is SEQ ID NO:35, with or without the C-terminal lysine. This constant region can be linked to any of the heavy chain variable regions. An exemplary light chain constant region is SEQ ID NO:30. This constant region can be linked to any of the light chain variable regions. The nucleic acids encoding the various heavy and light chains just described are provided in SEQ ID NOs:73-76 and 63-67.

Exemplary humanized forms of the mouse 1H7 antibody include four exemplified humanized light chain mature variable regions (Hu1H7VLv1-v4; SEQ ID NOs:109, 111, 113, and 115, respectively and five exemplified humanized heavy chain mature variable regions (Hu1H7VHv1-v5; SEQ ID NOs:95, 97, 99, 101, and 103, respectively). The exemplary light and heavy chain mature variable regions can be paired in any combination. An exemplary combination is Hu1H7VHv3 (SEQ ID NO:99) and Hu1H7VLv3 (SEQ ID NO:113). An exemplary heavy chain constant region is SEQ ID NO:35, with or without the C-terminal lysine. This constant region can be linked to any of the heavy chain variable regions. An exemplary light chain constant region is SEQ ID NO:30. This constant region can be linked to any of the light chain variable regions. An exemplified full length heavy chain has the amino acid sequence of SEQ ID NO:127 (with the G1m3). An exemplary full length light chain has the amino acid sequence of SEQ ID NO:125 (without Arginine). An exemplary combination is an antibody comprising SEQ ID NO:127 and SEQ ID NO:125. The nucleic acids encoding the various heavy and light chains just described are provided in SEQ ID NOs 108, 110, 112, 114, 94, 96, 98, 100, 102.

Such humanized antibodies are described in WO 06/020581 and U.S. Ser. Nos. 13/750,983, 14/049,169, 13/662,261, and 61/843,011.

In these or other antibodies described herein, one or more terminal residues can be cleaved in the course of posttranslational processing, particularly for terminal lysines of heavy chain constant regions.

### C. Chimeric and Veneered Antibodies

The invention further provides chimeric and veneered forms of non-human antibodies, particularly 9E4, 5C1 or 1H7.

A chimeric antibody is an antibody in which the mature variable regions of light and heavy chains of a non-human antibody (e.g., a mouse) are combined with light and heavy chain constant regions from an antibody of a different species. Typically, the light and heavy chain constant regions are of human origin, but the constant regions can originate from a different non-human species, such as a rat, as needed (e.g., to facilitate testing of the non-human antibody in an appropriate animal model). Such antibodies substantially or entirely retain the binding specificity of the non-human (e.g., mouse) antibody supplying the variable regions, and are about two-thirds human (or different non-human species) sequence.

A veneered antibody is a type of humanized antibody that retains some and usually all of the CDRs and some of the non-human variable region framework residues of a non-human antibody but replaces other variable region framework residues that may contribute to B- or T-cell epitopes, for example exposed residues (Padlan, Mol. Immunol. 28:489, 1991) with residues from the corresponding positions of a human antibody sequence. The result is an antibody in which the CDRs are entirely or substantially from a non-human antibody and the variable region frameworks of the non-human antibody are made more human-like by the substitutions. Veneered forms of 9E4 are included in the invention.

### D. Selection of Constant Region

The heavy and light chain variable regions of chimeric, veneered or humanized antibodies can be linked to at least a portion of a human constant region. The choice of constant region depends, in part, whether antibody-dependent cell-mediated cytotoxicity, antibody dependent cellular phagocytosis and/or complement dependent cytotoxicity are desired. For example, human isotopes IgG1 and IgG3 have complement-dependent cytotoxicity and human isotypes IgG2 and IgG4 do not. Human IgG1 and IgG3 also induce stronger cell mediated effector functions than human IgG2 and IgG4. Light chain constant regions can be lambda or kappa. An exemplary human light chain kappa constant region has the amino acid sequence of SEQ ID NO:13. Some such light chain kappa constant regions can be encoded by a nucleic acid sequence. The N-terminal arginine of SEQ ID NO:13 can be omitted, in which case light chain kappa constant region has the amino acid sequence of SEQ ID NO:30. Some such light chain kappa constant regions can be encoded by a nucleic acid sequence. An exemplary human IgG1 heavy chain constant region has the amino acid sequence of SEQ ID NO:15 (with or without the C-terminal lysine) or the heavy chain constant region component of SEQ ID NO:37 (with or without the C-terminal lysine). Some such heavy chain constant regions can be encoded by a nucleic acid sequence. Antibodies can be expressed as tetramers containing two light and two heavy chains, as separate heavy chains, light chains, as Fab, Fab′, F(ab′)2, and Fv, or as single chain antibodies in which heavy and light chain mature variable domains are linked through a spacer.

Human constant regions show allotypic variation and isoallotypic variation between different individuals, that is, the constant regions can differ in different individuals at one or more polymorphic positions. Isoallotypes differ from allotypes in that sera recognizing an isoallotype bind to a non-polymorphic region of a one or more other isotypes. Thus, for example, another heavy chain constant region is of IgG1 G1m3 allotype and has the amino acid sequence encoding a constant region of SEQ ID NO:37. Yet another heavy chain constant region has the amino acid sequence encoding a constant region of SEQ ID NO:37 except that it lacks the C-terminal lysine.

One or several amino acids at the amino or carboxy terminus of the light and/or heavy chain, such as the C-terminal lysine of the heavy chain, may be missing or derivatized in a proportion or all of the molecules. Substitutions can be made in the constant regions to reduce or increase effector function such as complement-mediated cytotoxicity or ADCC (see, e.g., Winter et al., U.S. Pat. No. 5,624,821; Tso et al., U.S. Pat. No. 5,834,597; and Lazar et al., Proc. Natl. Acad. Sci. USA 103:4005, 2006), or to prolong half-life in humans (see, e.g., Hinton et al., J. Biol. Chem. 279:6213, 2004). Exemplary substitutions include a Gln at position 250 and/or a Leu at position 428 (EU numbering is used in this paragraph for the constant region) for increasing the half-life of an antibody. Substitution at any or all of positions 234, 235, 236 and/or 237 reduce affinity for Fcγ receptors, particularly FcγRI receptor (see, e.g., U.S. Pat. No. 6,624,821). Some antibodies have alanine substitution at positions 234, 235 and 237 of human IgG1 for reducing effector functions. Optionally, positions 234, 236 and/or 237 in human IgG2 are substituted with alanine and position 235 with glutamine (see, e.g., U.S. Pat. No. 5,624,821).

### E. Expression of Recombinant Antibodies

Antibodies can be produced by recombinant expression. Nucleic acids encoding the antibodies can be codon-optimized for expression in the desired cell-type (e.g., CHO or Sp2/0). Recombinant nucleic acid constructs typically include an expression control sequence operably linked to the coding sequences of antibody chains, including naturally-associated or heterologous promoter regions. The expression control sequences can be eukaryotic promoter systems in vectors capable of transforming or transfecting eukaryotic host cells. Once the vector has been incorporated into the appropriate host, the host is maintained under conditions suitable for high level expression of the nucleotide sequences, and the collection and purification of the crossreacting antibodies. The vector or vectors encoding the antibody chains can also contain a selectable gene, such as dihydrofolate reductase, to allow amplification of copy number of the nucleic acids encoding the antibody chains.

E. coli is a prokaryotic host particularly useful for expressing antibodies, particularly antibody fragments. Microbes, such as yeast are also useful for expression. Saccharomyces is an exemplary yeast host, with suitable vectors having expression control sequences, an origin of replication, termination sequences and the like as desired. Typical promoters include 3-phosphoglycerate kinase and other glycolytic enzymes. Inducible yeast promoters include, among others, promoters from alcohol dehydrogenase, isocytochrome C, and enzymes responsible for maltose and galactose utilizations.

Mammalian cells can be used for expressing nucleotide segments encoding immunoglobulins or fragments thereof. See Winnacker, From Genes to Clones, (VCH Publishers, NY, 1987). A number of suitable host cell lines capable of secreting intact heterologous proteins have been developed in the art, and include CHO cell lines, various COS cell lines, HeLa cells, HEK293 cells, L cells, and non-antibody-producing myelomas including Sp2/0 and NS0. It can be advantageous to use nonhuman cells. Expression vectors for these cells can include expression control sequences, such as an origin of replication, a promoter, an enhancer (Queen et al., Immunol. Rev. 89:49 (1986)), and necessary processing information sites, such as ribosome binding sites, RNA splice sites, polyadenylation sites, and transcriptional terminator sequences. Suitable expression control sequences are promoters derived from endogenous genes, cytomegalovirus, SV40, adenovirus, bovine papillomavirus, and the like. See Co et al., J. Immunol. 148:1149 (1992).

Having introduced vector(s) encoding antibody heavy and light chains into cell culture, cell pools can be screened for growth productivity and product quality in serum-free media. Top-producing cell pools can then be subjected to FACS-based single-cell cloning to generate monoclonal lines. Specific productivities above 50 μg or 100 μg per cell per day, which correspond to product titers of greater than 7.5 g/L culture, can be advantageous. Antibodies produced by single cell clones can also be tested for turbidity, filtration properties, PAGE, IEF, UV scan, HP-SEC, carbohydrate-oligosaccharide mapping, mass spectrometery, and binding assay, such as ELISA or Biacore. A selected clone can then be banked in multiple vials and stored frozen for subsequent use.

Once expressed, antibodies can be purified according to standard procedures of the art, including protein A capture, column chromatography (e.g., hydrophobic interaction or ion exchange), low-pH for viral inactivation and the like (see generally, Scopes, Protein Purification (Springer-Verlag, NY, 1982)).

Methodology for commercial production of antibodies including codon optimization, selection of promoters, transcription elements, and terminators, serum-free single cell cloning, cell banking, use of selection markers for amplification of copy number, CHO terminator, serum free single cell cloning, improvement of protein titers (see, e.g., U.S. Pat. Nos. 5,786,464, 6,114,148, 6,063,598, 7,569,339, WO2004/050884, WO2008/012142, WO2008/012142, WO2005/019442, WO2008/107388, and WO2009/027471, and U.S. Pat. No. 5,888,809).

### V. Formulations

Formulations (also known as pharmaceutical compositions) of the invention comprise an antibody (e.g., a chimeric, veneered or humanized version of murine 9E4 (ATCC Accession Number PTA-8221)) or antigen binding fragment thereof, a buffer, one or more sugars and/or polyols and a surfactant, and have a pH within the range from about 5 to about 7.5. The formulations can be prepared for storage in liquid form or in lyophilized form. When stored in lyophilized form, the formulations can be reconstituted with a liquid (e.g., sterile water) to the concentrations and properties described herein. When a lyophilized composition is said to be reconstitutable by adding water to generate a formulation of specified component concentrations and pH, it is meant that the lyophilized formulation can be so reconstituted simply by addition of water (i.e., without supplying additional amounts of components or adding acid or base to change the pH). The concentrations and properties of a prelyophilized liquid formulation can also be in accordance with those described below if the lyophilized formulation is reconstituted to the same volume as the formulation prelyophilization. If the volume is different, then concentrations of formulations should be adjusted proportionally. For example, if the reconstituted volume is half the prelyophilization volume, then the concentrations of components in the prelyophilization formulation should be half the concentrations in the reconstituted formulation.

Some formulations include a bulking agent, which may or may not be the same as a sugar/polyol component. Typically, the formulations are sterile, for example, as accomplished by sterile filtration using a 0.2 μm or a 0.22 μm filter. Some formulations have a bioburden of ≤about 3 CFU/30 mL. Some formulations contain≤about 0.1 EU/mg of bacterial endotoxins. The formulations of the invention are also generally stable by low to undetectable levels of fragmentation and/or aggregation as further defined below on freezing and thawing. Still other formulations are stable following reconstitution of a lyophilized cake for at least three months at 40 degrees Celsius. In some formulations, less than about 10% of the antibody is present as an aggregate in the formulation. In some formulations, less than or equal to about 5% of the antibody is present as an aggregate in the formulation.

In some formulations, the antibody is present at a concentration within the range from about 5 mg/mL to about 100 mg/mL. In some formulations, the antibody is present at a concentration within the range from about 5 mg/mL to about 50 mg/mL. In some formulations, the antibody is present at a concentration within the range from about 25 mg/mL to about 50 mg/mL. For example, the antibody may be present at a concentration of about 35-45 mg/ml or about 40 mg/mL. The antibody may be present in a sterile liquid dosage form of about 50 mg/vial to about 500 mg/vial, or greater. The antibody may be present in a lyophilized dosage form of about 40 mg/vial to about 500 mg/vial. For example, the antibody may be present in a sterile liquid or lyophilized dosage form of about 250-350 mg/vial or about 200 mg/vial.

Antibodies used in the disclosed formulations can be coupled with a therapeutic moiety, such as a cytotoxic agent, a radiotherapeutic agent, an immunomodulator, a second antibody (e.g., to form an antibody heteroconjugate), or any other biologically active agent that facilitates or enhances the activity of the formulated antibody. However, antibodies are usually used in naked form (i.e., not conjugated to any of these moieties).

The formulated antibody can be any of the antibodies described above including any of the chimeric, veneered or humanized versions of antibody 9E4 described above.

Buffers are used in the disclosed formulations to achieve a suitable pH for the antibody, such as, for example, histidine, succinate, and citrate buffers. Some formulations have a pH within the range from about 5.5 to about 7, for example, a pH of 5.5, 5.6, 5.7, 5.8, 5.9, 6.0, 6.1, 6.2, 6.3, 6.4, 6.5, 6.6, 6.7, 6.8, 6.9, or 7.0. Some formulations have a pH of between about 5.5 to about 6.5. Some formulations have a pH of about 6.0 and other formulations have a pH of about 6.5. In some formulations, citrate buffer or succinate buffer is present at a concentration within the range from about 10 mM to about 30 mM, for example, at a concentration of about 15-25 mM or about 20 mM. Some citrate buffers comprise sodium citrate dehydrate and citric acid monohydrate at a concentration within the range from about 15 mM to about 20 mM and a range from about 2 mM to about 6 mM, respectively.

Suitable sugars and/or polyols for the formulations include trehalose, sucrose, mannitol, or a combination thereof. Sugars/polyols serves as bulking agents, lyoprotecting agent, and/or tonicity adjusting agents. For example, some formulations include trehalose present at a concentration within the range from about 220 mM to about 260 mM, sucrose present at a concentration within the range from about 220 mM to about 260 mM, or a mixture of sucrose present at a concentration within the range from about 20 mM to about 40 mM and mannitol present at a concentration within the range from about 200 mM to about 220 mM. Some formulations include trehalose present at a concentration of about 230 mM or 240 mM. Other formulations include sucrose present at a concentration of about 230 mM or 240 mM. Other formulations include a mixture of sucrose present at a concentration of about 50 mM and mannitol present at a concentration of about 200 mM. Another formulation includes a mixture of sucrose present at a concentration of about 28 mM and mannitol present at a concentration of about 212 mM. Some such formulations are characterized by an osmolality in the range of about 250-400, 300-400, or 300-350 mOsm/kg, such as, for example, 335 mOsm/kg.

Formulations can contain a surfactant to reduce antibody aggregation and absorption to surfaces. Suitable surfactants include polysorbate 20 (PS20) present at a concentration within the range from about 0.005% to about 0.05% by weight. PS20 protects against marked increases in aggregation or turbidity that would otherwise occur in formulations of 9E4 antibodies. The polysorbate 20 may be present at a concentration within the range from about 0.01% to about 0.05%. For example, the concentration can be 0.005%, 0.01%, 0.015%, 0.02%, 0.025%, 0.03%, 0.035%, 0.04%, 0.045%, or 0.05%. Alternatively, in some formulations, polysorbate 20 is present at a concentration within the range of about from about 0.05 g/L, 0.1 g/L, 0.15 g/L, 0.2 g/L, 0.25 g/L, 0.3 g/L, 0.35 g/L, 0.4 g/L, 0.45 g/L, or 0.5 g/L. Some formulations include polysorbate 20 at a concentration of 0.2 g/L (i.e., 0.163 mmol/L).

An exemplary formulation (liquid, prelyophilization or reconstituted after lyophilization) is characterized by a pH within the range from about 5.5 to about 7 and includes: (a) a chimeric, veneered, or humanized version of antibody 9E4, or a fragment thereof that specifically competes for binding to antigen with 9E4 at a concentration within the range from about 10 mg/ml to about 50 mg/ml; (b) a citrate buffer or succinate buffer present at a concentration within the range from about 10 mM to about 30 mM; (c) one or more sugars and polyols (“sugar/polyol”) selected from trehalose present at a concentration within the range from about 220 mM to about 260 mM, sucrose present at a concentration within the range from about 220 mM to about 260 mM, and a mixture of sucrose present at a concentration within the range from about 20 mM to about 40 mM and mannitol present at a concentration within the range from about 200 mM to about 220 mM; and (d) polysorbate 20 present at a concentration within the range from about 0.005% to about 0.05% by weight. For example, the formulation can include: (a) an antibody comprising a light chain having the amino acid sequence set forth as SEQ ID NO: 32 and a heavy chain comprising an amino acid sequence set forth as SEQ ID NO: 37, with or without the C-terminal lysine, and which is present at a concentration of about 40 mg/mL; (b) a citrate buffer at a concentration of about 20 mM; (c) trehalose at a concentration of about 230 mM; (d) polysorbate 20 at a concentration of about 0.02%; and a pH of about 6.0.

Some lyophilized formulations include: (a) a humanized version of antibody 9E4 or an antigen binding fragment thereof; (b) citrate; (c) trehalose; and polysorbate 20. The lyophilized formulation can include about 200 mg of the antibody. Some lyophilized formulations are capable of being reconstituted with sterile water. Some lyophilized formulations include 100-300 or 150-250 mg 9E4 antibody, 15-35 or 20-25 mg sodium citrate dehydrate, 1.65-2.75 or 2-2.3 mg citric acid monohydrate, 360-500 or 400-470 mg trehalose dehydrate, and 0.5 to 1.5 mg or 0.75 to 1.25 mg polysorbate 20. An exemplary lyophilized formulation includes 200 mg of a 9E4 antibody (e.g., humanized 9E4 antibody), 25 mg of sodium citrate dehydrate, 2.15 mg citric acid monohydrate, 435 mg trehalose dehydrate, and 1 mg polysorbate 20. Another exemplary lyophilized formulation includes 200 mg of a 9E4 antibody (e.g., humanized 9E4 antibody), 25 mg of sodium citrate dehydrate, 3.15 mg citric acid monohydrate, 435 mg trehalose dehydrate, and 1 mg polysorbate 20. Such formulations can be reconstituted to a volume of about 5 ml. Other lyophilized formulations include the same components in the same proportions as any disclosed in this paragraph but in different amounts (e.g., 400 mg antibody, 50 mg sodium citrate, 4.3 mg citric acid monohydrate, 870 mg Trehalose dehydrate, and 2 mg polysorbate 20).

Lyophilized formulations can be reconstituted to an antibody concentration of about 30-50 or 35-45 mg/mL, such as about 40 mg/mL; (b) a citrate buffer present at a concentration of about 10-30 or 15-25 mM, preferably about 20 mM; (c) trehalose present at a concentration of about 160-330 or 200-260 mM, such as about 230 mM; (d) polysorbate 20 present at a concentration of about 0.1-0.3 or 0.15 to 0.25 g/L, such as about 0.2 g/L; and (e) a pH of about 5.5-6.5, such as about 6.0.

Liquid or reconstituted lyophilized formulations can be substantially isotonic, implying an osmolality of about 250-350 mOsm/kg water. Some formulations have an osmolality of about 335 mOsm/kg. Some formulations have an osmolality of 270-300 mOsm/kg. Liquid or reconstituted lyophilized formulations can also be hypertonic>350 mOsm/kg water or hypotonic (<250 mOsm/kg water).

Any of the formulations described can be made without pharmaceutical excipients, carriers or the like, other than those described as being components herein. Such a formulation can be described as consisting of the recited components, or consisting essentially of the recited components if insignificant amounts of other components not affecting the properties of the formulation are present. Formulations can be made under good manufacturing practices (GMP) approved or approvable by the FDA for preparation of drugs for administration to humans.

### VI. Diagnostic Criteria for Synucleinopathies

The present methods are in general performed on subjects diagnosed with a Lewy body disease by a qualified health practitioner or are at elevated risk thereof compared with the general population as evidenced by genetic or biochemical markers, family history or prodromal symptoms of the disease. Such individuals include any who have received a prior prescription for treatment or prophylaxis of a Lewy body disease. Diagnosis of the synucleinopathy can be based on art-recognized criteria for possible or probable Lewy body disease, such as those of DSM-V or DSM IV-TR, the Lewy Body dementia association, the Parkinson's disease society and the like. However, diagnosis can also be based on presence of any signs or symptoms of Lewy body disease that lead a treating physician to conclude that a subject probably has a Lewy body disease. Exemplary criteria for diagnosing possible or probable PD are shown below.
Group A: resting tremor, bradykinesia, rigidity and asymmetric onset
Group B features: suggestive of alternative diagnoses
   Prominent postural instability in the first 3 years after symptom onset
   Freezing phenomena in the first 3 years
   Hallucinations unrelated to medications in the first 3 years
   Dementia preceding motor symptoms or in the first year
   Supranuclear gaze palsy (other than restriction of upward gaze) or slowing of vertical saccades
   Severe symptomatic dysautonomia unrelated to medications

Documentation of a condition known to produce parkinsonism and plausibly connected to the subject's symptoms (such as suitably located focal brain lesions or neuroleptic use within the past 6 months).

Criteria for possible diagnosis of Parkinson disease:
At least 2 of the 4 features in Group A are present; at least 1 of these is tremor or bradykinesia and either none of the features in group B is present or symptoms have been present for less than 3 years and none of the features in group B is present to date; and either substantial and sustained response to levodopa or a dopamine agonist has been documented, or the subject has not had an adequate trial of levodopa or dopamine agonist.

Criteria for probable diagnosis of Parkinson disease:
At least 3 or the 4 features in Group A are present, and none of the features in Group B is present and substantial and sustained response to levodopa or a dopamine agonist has been documented.

Exemplary criteria for diagnosis of Lewy Body dementia are:
The presence of dementia
At least two of three core features:
   fluctuating attention and concentration,
   recurrent well-formed visual hallucinations, and
   spontaneous parkinsonian motor signs.
Suggestive clinical features include:
   Rapid eye movement (REM) sleep behavior disorder
   Severe neuroleptic sensitivity
   Low dopamine transporter uptake in basal ganglia demonstrated by SPECT or PET imaging

In the absence of two core features, the diagnosis of probable DLB can also be made if dementia plus at least one suggestive feature is present with one core feature.

Possible DLB can be diagnosed with the presence of dementia plus one core or suggestive feature.

Early warning signs of Lewy Body disease include for example, EEG slowing, neuropsychiatric manifestations (depression, dementia, hallucinations, anxiety, apathy, anhedonia), autonomic changes (orthostatic hypotension, bladder disturbances, constipation, fecal incontinence, sialorrhea, dysphagia, sexual dysfunction, changes in cerebral blood flow), sensory changes (olfactory, pain, color discrimination abnormal sensations), sleep disorders (REM sleep behavior disorder (RBD), restless legs syndrome/periodic extremity movements, hypersomnia, insomnia) and miscellaneous other signs and symptoms (fatigue, diplopia, blurred vision, seborrhea, weight loss/gain). Genetic markers of risk toward PD include mutations in the alpha-synuclein or Parkin, UCHLI, and CYP2D6 genes; particularly mutations at positions 30 and 53 of the alpha-synuclein gene. None of these genetic markers or early warning sign is itself diagnostic of a Lewy body disease but they can individually or in combination contribute to a diagnosis of a Lewy body disease.

### VII. Therapeutic Regimes

In therapeutic applications, an antibody is administered to a subject diagnosed with a synucleinopathy in a regime (dose, frequency and route of administration) known or suspected to be effective to ameliorate or at least inhibit further deterioration of at least one sign or symptom of the disease. In prophylactic applications, an antibody is administered to a subject at increased risk of a synucleinopathy but not yet having sufficient symptoms to be diagnosed with the disease in a regime known or suspected to be effective to inhibit or delay onset of at least one sign or symptom of the disease.

A regime is considered therapeutically or prophylactically effective if a more favorable outcome is demonstrated in treated subjects versus control subjects in a controlled animal model or clinical trial (e.g., a phase II, phase II/III or phase III trial) at the p<0.2, 0.1, 0.05 or 0.01 or even 0.001 level. However, due to variations in genetics, subject characteristic and environment and disease subtype between individual subjects, a regime that is effective in one subject may not be effective in another subject or may be effective to different extents.

An exemplary dosage range for antibodies is from 3000 to 5000 mg of an antibody against alpha-synuclein administered intravenously at intervals of 3-5 weeks, such as every 4 weeks. In some subjects, the dosage is 3500-4500 mg every 3-5 weeks, such as every 4 weeks. Subjects can receive the same or different dosages as each other (e.g., depending on weight of the subject). In some methods, a subject receives one of two fixed dosages. For example, subjects with a weight less than 65 kg can receive 3500 mg and subjects with a weight greater or equal to 65 kg can receive 4500 mg. In some methods, the dosage range for at least some subjects lies within a range of 45-75 mg/kg, for example, 50-70 mg/kg, 45 mg/kg, 60 mg/kg or 65 mg/kg. Dosages are usually administered on multiple occasions with an interval of 3-5 weeks, such as every 28 days or four weeks, or every calendar month. Subjects can receive at least 6, 9, 12 or 18 dosages at such intervals, or can be dosed while symptoms of the conditions persist or for the remaining life of the subject. In some regimes, an initial loading dose of 2000 mg is administered followed by dosing within a range of greater or equal to 2000 mg but less than the intended target dose until the intended target dose is reached. For example, a subject can receive an initial loading dose of 2000 mg, followed by an up-titration to a 3500 mg dose or a 4500 mg dose. The up-titration can occur in a single subsequent dose or in gradual increases over several doses until a target dose or dose within a target range is reached. For example, a subject can receive an initial dose of 2000 mg followed by subsequent doses of 3500 mg. Alternatively a subject can receive an initial dose of 2000 mg followed by one or more subsequent doses at greater or equal to 2000 mg but less than 3500 mg, and subsequent doses at 3500 mg. Likewise a subject can receive an initial dose of 2000 mg followed by subsequent doses of 4500 mg. Alternatively, a subject can receive an initial dose of 2000 mg followed by subsequent doses at greater or equal to 2000 mg but less than 4500 mg and subsequent doses at 4500 mg. In some regimes a subject receives a dose of 3000-5000 mg antibody intravenously every four weeks for at least 52 weeks. In subjects receiving multi-dose regimes with the dose within a specified range, such as 3500-5000 mg, the subject can receive the same or different dose within the specified range on each dosing. In some regimes, a subject receives the same dose within a specified range at each dosing.

In another exemplary regime, a dose of 1300-1700 mg antibody is administered intravenously to a subject at intervals of 3-5 weeks. An exemplary dose is 1500 mg. Subjects can received a single fixed dose or two or more different dosages within this range, based on e.g., subject weight. Some subjects dosed within this range receive 18-25 mg/kg of antibody, for example, 20 mg/kg. As in other methods, the intervals can be 3-5 weeks, such as every 4 weeks or every calendar month. Subjects can receive at least 6, at least 9, at least 12, or at least 18 dosages, or can be dosed at such intervals while symptoms remain or for the remaining life of a subject.

For any of the above described treatment regimes, an area under the curve can be calculated to indicate the amount of antibody delivered with time. Alternative treatment regimes can be devised (e.g., with a different route of administration, frequency or dose) to deliver substantially the same area under the curve (e.g., within 25, 20 or 15%). Preferably such an alternative treatment regime does not substantially exceed the Cmax of the specified regime (e.g., by no more than 25, 20 or 15%). Other routes of administration include topical, oral, subcutaneous, intraarterial, intracranial, intrathecal, intraperitoneal, intranasal or intramuscular.

Any of the treatment regimes can be accompanied by monitoring a subject receiving treatment for changes in movement and/or cognitive deficits. Preferably such monitoring includes at least one assessment before and after commencing treatment. Preferably the monitoring indicates reduced movement and/or cognitive deficits responsive to treatment, that is relative to before beginning treatment or at least indicates a reduced rate of decline relative to the previous rate of decline in the subject or the rate of decline in control patients not receiving any immunotherapy. Subjects can also be monitored for changes in autonomic dysfunction, gastrointestinal dysfunction, visual hallucination or one or more psychological symptom among other signs or symptoms.

Symptoms of subjects, for example motor symptoms such as tremor, rigidity and slowness of movement, may be monitored. Wearable systems or on-body sensors may be used to assess and quantify motor symptoms of subjects. “On-body sensors” may be used in a laboratory setting or in free-living conditions [S. Del Din, et al., J. of NeuroEngineering and Rehabilitation, 2016 13:46].

Subjects may be monitored using mobile-device-based monitoring. The mobile device may be a smartphone, smartwatch, wearable sensor, portable multimedia device or tablet computer. Built-in mobile-device sensors may be used to record daily activities of subjects. Subjects may carry a mobile-device to record their daily activities. Mobile-device-based assessments and sensors may be used for remote, passive monitoring of gait and mobility in subjects receiving treatment, for example for Parkinson's disease. (See e.g., Lipsmeier, F., et al., Mov Disord. 2017; 32 (suppl 2); W. Y. Cheng et al., 2017 IEEE/ACM International Conference on Connected Health: Applications, Systems and Engineering Technologies (CHASE), Philadelphia, PA, 2017, pp. 249-250). Sensor data may be analyzed by machine learning-based activity profiling. Gait and mobility may be correlated with the MDS-UPDRS that is used in clinics to evaluate Parkinson's disease severity.

Some mobile-device-based monitoring may comprise (a) providing a subject with a mobile device programmed to receive and transmit data acquired from sensors internal and/or external to the device relating to movement deficits of a subject having or suspected of having a synucleinopathy, whereafter the subject undergoes a series of movements to reveal movement deficits, if present, and the internal or external sensors of the device acquire data relating to the movements; (b) collecting data transmitted from the mobile device; and (c) comparing the data acquired from the subjects with control data to assess presence or extent of movement deficits in the subject. In some mobile-device-based monitoring, the mobile device is programmed to receive and transmit data from at least two external sensors attached to upper and lower limbs of the subject. In some mobile-device-based monitoring, the mobile device acquires data from sensors on the upper and lower limbs of the subject. In some mobile-device-based monitoring, the mobile device is carried by the subject and acquires data from an internal sensor. In some mobile-device-based monitoring, the series of movements includes tapping the device, sitting and standing.

The present regimes can be administered concomitantly with another agent effective in treatment or prophylaxis of the disease being treated. The other agent can be another immunotherapeutic agent described herein or other agent for treating Parkinson's disease including levodopa, benzaseride, carbidopa, dopamine agonists, non-ergot dopamine agonists, catechol-O-methyl (“COMT”) inhibitors such as, for example, entacopone or tolcopone, monoamine oxidase (“MAO”) inhibitors, such as, for example, rasagaline, amantadine, or anticholinergic agents can be used in combination with the present regimes. Some such other agents reduce one or more symptoms of the disease without affecting causative factors.

All publications (including GenBank Accession numbers, UniProtKB/Swiss-Prot accession numbers and the like), patents and patent applications cited are herein incorporated by reference in their entirety for all purposes to the same extent as if each individual publication, patent and patent application was specifically and individually indicated to be incorporated by reference in its entirety for all purposes. In the event of any variance in sequences associated with Genbank and UniProtKB/Swiss-Prot accession numbers and the like or in website, or disease criteria of an organization, the application refers to those in effect on its effective filing date.

### VIII. EXAMPLES

### Example 1. Phase II Clinical Trial for an Alpha-Synuclein Antibody

Trial Design: A phase II trial is conducted for an alpha-synuclein antibody with a heavy chain variable region designated SEQ ID NO:10 and a light chain variable region designated SEQ ID NO:5 on subjects with Parkinson's disease. The trial has two treatment arms and one control arm. Subjects are randomized 1:1:1 into the arms, with N=300. The initial phase of the trial is a 52-week double blind treatment. During the initial phase of the trial, subjects do not receive other treatments Parkinson's disease (including symptomatic treatment). The subjects in one treatment arm receive a fixed dose of 1500 mg antibody intravenously every four weeks. The subjects in the other treatment arm receive 3500 mg or 4500 mg of antibody intravenously every four weeks depending on weight with subjects below 65 kg receiving the lower dosage and subjects at or above 65 kg receiving the higher dosage. The subjects in the second arm receive a loading dosage of 2000 mg and optionally additional up titration dosages at 2000 mg or above until reaching the target dose of 3500 mg or 4500 mg. Dosing is continued for one year. The trial then has an extension period in which subjects initially in the placebo group receive one of the two treatment regimes from the initial phase, and subjects from the treatment arms in the initial phase continue to receive the same treatment as previously. During the extension phase of the trial, subjects may receive systematic treatment with levodopa as well as the antibody subject of the trial, but do not receive other treatments for Parkinson's disease.

### Objectives:

The primary objective is to evaluate efficacy of an alpha-synuclein antibody with a heavy chain variable region designated SEQ ID NO:10 and a light chain variable region designated SEQ ID NO:5 using the Movement Disorder Society (MDS)-sponsored revision of the Unified Parkinson's Disease Rating Scale (MDS-UPDRS).

Secondary objectives are to:
evaluate the effects of the antibody on DaT-SPECT signal and signs and symptoms of Parkinson's disease including changes in movement and cognitive deficits
evaluate safety and tolerability of the antibody for up to 104 weeks, and
evaluate pharmacokinetics of the antibody.

### Example 2. Passive Monitoring of Early-Stage Parkinson's Disease Patient Mobility in Phase I Alpha-Synuclein Antibody Clinical Trial with Smartphone Sensors

1. Abstract—Gait and mobility in early-stage Parkinson's disease (PD) patients were measured using smartphone-based passive monitoring. In the Multiple Ascending Dose clinical trial of an alpha-synuclein antibody with a heavy chain variable region designated SEQ ID NO:10 and a light chain variable region designated SEQ ID NO:5, 44 PD patients and 35 age- and gender-matched healthy individuals performed smartphone-based assessments for up to 24 weeks and up to 6 weeks respectively. (Lipsmeier, F., et al., Mov Disord. 2017; 32 (suppl 2); W. Y. Cheng et al., 2017 IEEE/ACM International Conference on Connected Health: Applications, Systems and Engineering Technologies (CHASE), Philadelphia, PA, 2017, pp. 249-250).

For “passive monitoring”, subjects carried the smartphone with them as part of their daily routine, while sensors in the smartphone recording movement data continuously. In total, over 30,000 hours of passive monitoring data were collected. To classify the sensor signal into activity profiles, a Human Activity Recognition (HAR) model was built using Deep Neural Networks (DNN) trained on previously published data. The activity profiles of the participants determined by the HAR model showed significant differences between PD patients and healthy controls in the percentage of time walking and frequency in which subjects changed positions (sitting and standing).

### 2. Methods

### A. Data Collection

The analysis solely focused on exploring differences between HC and PD cohorts, and did not look at antibody-related effects. In total, 24,104 hours of passive monitoring data were recorded for the PD cohort, and 8,614 hours for the HC cohort. In line with the approach of Rai, A. et al., (MobiCom'12, Aug. 22-26, 2012], accelerometer data was filtered out where the standard deviation of Euclidean norm less than 0.03 m/s2 more than 30 minutes, as during these spans smartphones were likely not carried by the subjects. This step removed 14% of the passive monitoring data.

### B. HAR

A 9-layer neural network model structure was used. Similar structures have been used previously for HAR and have been shown to out-perform the traditional machine learning methods (F. J. Ordóñez and D. Roggen, Sensors 2016, 16, 115]. The HAR model was trained on two public data sets (G. M. Weiss and J. W. Lockhart, Proceedings of the AAAI-12 Workshop on Activity Context Representation: Techniques and Languages, Toronto, CA. 2012; A. Stisen, et al., 13th ACM Conference on Embedded Networked Sensor Systems, Seoul, Korea, 2015) to classify six activities: walking, stairs, jogging, sitting, standing, and lying down. The continuous accelerometer data were down-sampled into 20 Hz and segmented into 4-second windows with 75% overlapping with adjacent ones.

### 3. Results

### A. Human Activity Recognition Performance Validation:

To ensure the HAR model can accurately translate the sensor data into activity profile, the performance of the model was first analyzed in the held-out validation set. The HAR model was able to correctly distinguish gait activities (walking, stairs, jogging) from stationary activities (sitting, standing, lying down) with more than 98% of accuracy. Additional validation on labeled Gait and Balance data from the trial data also showed that the HAR model was able to successfully profile the Gait segments with 96.9% of accuracy, and Balance segments with 99.5% accuracy.

### B. Activity Profiles Comparison

The mobility of each subject was quantified by calculating the proportion of time when the subject engaged in gait activities (walking, stairs, jogging) over the total passive monitoring coverage time of the patient. The overall proportion of different gait activities over the total coverage for PD and HC cohorts was calculated. In the PD cohort a median was detected of 9.7% of gait spans over all coverage spans as opposed to HC cohort's 15.1%. The HC cohort had a significantly higher per-subject gait activity level than PD cohort, with Mann-Whitney test P value 2.43E-8.

### C. Number of Sit-to-Stand and Stand-to-Sit Comparison

It has been observed that one manifestation of the functional impact of PD is in the sit-to-stand and stand-to-sit (STS) events (A. Zijlstra, et al., J. NeuroEngineering and Rehabilitation 2012, 9:75]. From the activity profile, the coverage-normalized STS events were calculated for each subject. Median number of STS per hour of PD patient of 1.44 was observed, which was significantly lower than HC subject's 1.74. Mann-Whitney test P value between two groups was 1.60E-8.

### 4. Conclusion

Results from this study show that it is feasible to measure gait and mobility in early-stage PD patients using smartphone-based passive monitoring. Sensor data collected during passive monitoring provides previously inaccessible, ecologically valid insights into patients' daily behavior and functioning. Significant differences were observed between PD patients and healthy controls (HC).

1-107. (canceled)

108. A method of treating or effecting prophylaxis of a subject having a Lewy body disease, the method comprising intravenously administering to the subject a dose of 1300-1700 mg of an antibody or an antigen binding fragment thereof against alpha-synuclein at intervals of 3-5 weeks, or administering another regimen that delivers the antibody or the antigen binding fragment thereof to the subject with substantially the same area under the curve.

109. The method of claim 108, wherein the subject receives a dose of 18-25 mg/kg.

110. The method of claim 108, wherein the dose is about 1512 mg.

111. The method of claim 108, wherein the subject receives between at least 6 doses to at least 18 doses of the antibody or the antigen binding fragment thereof at intervals of 3-5 weeks.

112. The method of claim 108, wherein the interval is 4 weeks.

113. The method of claim 108, wherein the subject receives the antibody or the antigen binding fragment thereof every 4 weeks for at least 52 weeks.

114. The method of claim 113, wherein the subject has early-stage Parkinson's disease.

115. The method of claim 108, further comprising monitoring the subject for a change in movement, cognitive deficit autonomic dysfunction, gastrointestinal dysfunction, visual hallucination or a psychological symptom.

116. The method of claim 115, wherein the monitoring comprises:
(a) providing a subject with a mobile device programmed to receive and transmit data acquired from sensors internal and/or external to the device relating to movement deficits of a subject having or suspected of having a Lewy body disease, whereafter the subject undergoes a series of movements to reveal movement deficits, if present, and the internal or external sensors of the device acquire data relating to the movements;
(b) collecting data transmitted from the mobile device; and
(c) comparing the data acquired from the subjects with control data to assess presence or extent of movement deficits in the subject.

117. The method of claim 116, wherein the mobile device is programmed to receive and transmit data from at least two external sensors attached to upper and lower limbs of the subject.

118. The method of claim 116, wherein the mobile device is carried by the subject and acquires data from an internal sensor.

119. The method of claim 108, wherein the antibody or the antigen binding fragment thereof binds within residues 115-130 of alpha-synuclein.

120. The method of claim 108, wherein the antibody or the antigen binding fragment thereof comprises three heavy CDRs designated SEQ ID NOs:139-141 respectively and three light chain CDRs designated SEQ ID NOs:143-145 respectively.

121. The method of claim 108, wherein the antibody or the antigen binding fragment thereof comprises a heavy chain variable region comprising SEQ ID NO:138 and a light chain variable region comprising SEQ ID NO:142.

122. The method of claim 108, wherein the antibody or the antigen binding fragment thereof comprises the three light chain Kabat CDRs of SEQ ID NO:5 and the three heavy chain Kabat CDRs of SEQ ID NO:10.

123. The method of claim 108, wherein the antibody or the antigen binding fragment thereof comprises a heavy chain variable region comprising any one of SEQ ID NOs:8-11 and a light chain variable region comprising any one of SEQ ID NOs:3-5.

124. The method of claim 108, wherein the antibody or the antigen binding fragment thereof comprises a heavy chain variable region comprising SEQ ID NO:10 and a light chain variable region comprising SEQ ID NO:5.

125. The method of claim 108, wherein the antibody or the antigen binding fragment thereof is of human IgG1 isotype.

126. The method of claim 125, wherein the antibody or the antigen binding fragment thereof comprises a heavy chain constant region comprising SEQ ID NO:35 provided the C-terminal lysine may be absent and a light chain constant region comprising SEQ ID NO:30.

127. The method of claim 125, wherein the antibody or the antigen binding fragment thereof comprises a heavy chain constant region comprising SEQ ID NO:35 provided the C-terminal lysine may be absent and a light chain constant region comprising SEQ ID NO:13.

128. The method of claim 108, wherein the antibody or the antigen binding fragment thereof comprises three heavy CDRs designated SEQ ID NOs:146-148 respectively and three light chain CDRs designated SEQ ID NOs:149-151, respectively.

129. The method of claim 108, wherein the antibody or the antigen binding fragment thereof comprises a heavy chain comprising SEQ ID NO:37 and a light chain comprising SEQ ID NO:32, wherein the C-terminal lysine of SEQ ID NO:37 may be absent.

130. The method of claim 108, wherein the antibody or antigen binding fragment thereof binds within residues 1-20 of alpha-synuclein.

131. The method of claim 108, wherein the antibody or the antigen binding fragment thereof is NI-202.12F4 or NI-202.21D11.

132. The method of claim 108, wherein the antibody or the antigen binding fragment thereof comprises three heavy CDRs designated SEQ ID NOs:131-133 respectively and three light chain CDRs designated SEQ ID NOs:135-137 respectively.

133. The method of claim 108, wherein the antibody or the antigen binding fragment thereof comprises a heavy chain variable region comprising SEQ ID NO:130 and a light chain variable region comprising SEQ ID NO:134.

134. The method of claim 108, wherein the Lewy body disease is Parkinson's disease, dementia with Lewy bodies, multiple system atrophy, progressive supra nuclear palsy, REM sleep behavior disorder, or Alzheimer's disease with amygdala Lewy bodies.

135. The method of claim 108, wherein the subject is not receiving symptomatic treatment for Parkinson's disease concomitant with the antibody or the antigen binding fragment thereof.

136. The method of claim 108, wherein the antibody or the antigen binding fragment thereof is administered concomitantly with levodopa.

137. The method of claim 108, wherein administering the antibody or the antigen binding fragment thereof is preceded by administering a loading dose of 2000 mg of the antibody or the antigen binding fragment thereof and optionally uptitration at one or more subsequent dose at greater or equal to 2000 mg but less than 3500 mg until a dose of 3500 mg is reached, all doses being separated by intervals of 3-5 weeks.
